(19) United States
(12) Patent Application Publication
(10) Pub. No.: US 2014/0296099 A1
(43) Pub. Date: Oct. 2, 2014

(54) Use of MicroRNA for Assessing Embryos Grown in Vitro and Improving Culture Media

(71) Applicant: UNIVERSITY OF IOWA RESEARCH FOUNDATION, IOWA CITY, IA (US)

(72) Inventors: Bradley Van Voorhis, Iowa City, IA (US); Evan Rosenbluth, Iowa City, IA (US)

(73) Assignee: UNIVERSITY OF IOWA RESEARCH FOUNDATION, IOWA CITY, IA (US)

(21) Appl. No.: 14/250,984

(22) Filed: Apr. 11, 2014

Related U.S. Application Data
(63) Continuation-in-part of application No. PCT/US2012/059885, filed on Oct. 12, 2012.
(60) Provisional application No. 61/663,792, filed on Jun. 25, 2012.
(60) Provisional application No. 61/546,320, filed on Oct. 12, 2011.

(51) Int. Cl. C12Q 1/68; C12N 5/073

(52) U.S. Cl. CPC C12Q 1/6888; C12N 5/0604
USPC 506/9; 435/6.12; 435/375; 506/16; 435/6.11; 600/35

(57) ABSTRACT

Disclosed are methods of detecting miRNA expression by an embryo. The methods may be utilized for assessing embryo viability and chromosomal makeup and selecting the embryo for subsequent implantation into a patient. The methods also may be utilized for modifying and optimizing culture media to improve embryo viability via adding one or more miRNAs to culture media for the embryo or depleting one or more miRNAs from the culture media for the embryo. Kits for performing the disclosed methods are also disclosed.

### CROSS-REFERENCE TO RELATED PATENT APPLICATIONS

The present application is a continuation-in-part of international application number PCT/US2012/059885, filed on Oct. 12, 2012, which claims priority to U.S. Provisional Patent Application No. 61/663,792, filed on Jun. 25, 2012, and U.S. Provisional Patent Application No. 61/546,320, filed on Oct. 12, 2011, the contents of which are incorporated herein by reference in their entireties.

### FIELD

The field of the invention relates to microRNAs and the use thereof for assessing viability of embryos grown in vitro, assessing the chromosomal makeup of embryos grown in vitro, and for improving culture media for embryos grown in vitro. In particular, the field of the invention relates to the analysis of expressed microRNA for assessing embryonic health and likelihood for successful implantation, determining genotype of the embryo or the chromosomal makeup embryo, and optimizing culture media for growing embryos by modifying the concentration of microRNA in the media.

### BACKGROUND

MicroRNAs (miRNAs), also known as “mature miRNA” are small (approximately 18-24 nucleotides in length), non-coding RNA molecules encoded in the genomes of plants and animals. In certain instances, highly conserved, endogenously expressed miRNAs regulate the expression of genes by binding to the 3′-untranslated regions (3′-UTR) of specific mRNAs. More than 1000 different miRNAs have been identified in plants and animals. Certain mature miRNAs appear to originate from long endogenous primary miRNA transcripts (also known as pri-miRNAs, pri-mirs, pri-miRs or pri-pre-miRNAs) that are often hundreds of nucleotides in length (Lee, et al., EMBO J., 2002, 21(17), 4663-4670).

Functional analyses of miRNAs have revealed that these small non-coding RNAs contribute to different physiological processes in animals, including developmental timing, organogenesis, differentiation, patterning, embryogenesis, growth control and programmed cell death. Examples of particular processes in which miRNAs participate include stem cell differentiation, neurogenesis, angiogenesis, hematopoiesis, and exocytosis (reviewed by Alvarez-Garcia and Miska, Development, 2005, 132, 4653-4662). Here, miRNAs have been identified that are associated with embryo viability and chromosomal makeup.

### SUMMARY

Disclosed are methods for utilizing miRNAs in the culture and selection of embryos grown in vitro for subsequent implantation into a patient. The methods may include detecting expression of miRNAs in order to assess embryos grown in culture media in vitro and for improving culture media for embryos grown in vitro. Also disclosed are kits that include reagents for performing the disclosed methods.

The disclosed methods may include detecting intracellular expression of one or more miRNAs of an embryo grown in culture media in vitro. In other embodiments, the methods may include detecting extracellular expression of one or more miRNAs of an embryo grown in culture media in vitro. Detecting expression of the one or more miRNAs by the embryo may include, but is not limited to contacting a sample of the culture media or a sample of the embryo with a reagent that detects the one or more miRNAs. For example, the reagent may include an oligonucleotide that hybridizes to the one or more miRNAs, such as a DNA oligonucleotide that is utilized as a primer for performing RT-PCR where the miRNA, if present in the sample, functions as a template for RT-PCR.

The methods may include detecting intracellular and/or extracellular expression of one or more miRNAs of an embryo grown in vitro. Preferably, prior to performing the methods, the embryo has been grown in vitro for at least 1, 2, 3, 4, 5, or more days. In some embodiments, prior to performing the methods, the embryo may have been grown in the culture media for a sufficient period of time for the embryo to develop into a blastocyst.

The embryos for use in the disclosed methods may have been obtained by fertilization of an oocyte. In some embodiments, the embryos utilized in the disclosed methods are formed from oocytes that have been fertilized via intracytoplasmic sperm injection (ICSI). In other embodiments, the embryos utilized in the disclosed methods are formed from an oocyte that has been fertilized by standard, regular, or natural contact with a sperm cell without ICSI.

The disclosed methods may be performed in order to detect one or more miRNAs. Suitable miRNAs for the disclosed methods may include, but are not limited to hsa-miR-17, hsa-miR-19, has-miR-19a, hsa-miR-19b, hsa-miR-20a, hsa-miR-24, hsa-miR-25, hsa-miR-26b, hsa-miR-26b#, hsa-miR-27b, hsa-miR-29b, hsa-miR-29b-1, hsa-miR-29b-2, hsa-miR-30a-5p, hsa-miR-30b, hsa-miR-30c, hsa-miR-30c-1, hsa-miR-30c-2, hsa-miR-31, hsa-miR-92a, hsa-miR-93, hsa-miR-106, hsa-miR-106a, hsa-miR-106b, hsa-miR-140-5p, hsa-miR-141, hsa-miR-146b-5p, has-miRNA-148a, hsa-miR-149, hsa-miR-151-3p, hsa-miR-151-5p, has-155, hsa-miR-182#, hsa-miR-191, hsa-miR-192, hsa-miR-193b, hsa-miR-200c, hsa-miR-206, hsa-miR-302a, hsa-miR-302a#, hsa-miR-302b, hsa-miR-302b#, hsa-miR-302c, hsa-miR-302d, hsa-miR-320, hsa-miR-339, hsa-miR-339-3p, hsa-miR-345, hsa-miR-346, hsa-miR-347, hsa-miR-362-3p, hsa-miR-367, hsa-miR-371-3p, hsa-miR-372, hsa-miR-373, hsa-miR-374a, hsa-miR-376a, hsa-miR-376a, hsa-miR-376a-1, hsa-miR-376a-2, hsa-miR-378, has-miR-380-5p, hsa-miR-454, hsa-miR-484, hsa-miR-487b, hsa-miR-494, hsa-miR-500, hsa-miR-509-5p, hsa-miR-512-1, hsa-miR-512-2, hsa-miR-512-3p, hsa-miR-512-5p, hsa-miR-517c, hsa-miR-518a-1, hsa-miR-518a-2, hsa-miR-518a-3p, hsa-miR-518c, hsa-miR-518d, hsa-miR-518d-5p, hsa-miR-518e, hsa-miR-519a, hsa-miR-520c-3p, hsa-miR-520D-3p, hsa-miR-520d-5p, hsa-miR-522, hsa-miR-525-3p, hsa-miR-548a-3p, hsa-miR-548c-3p, hsa-mir-548d-3p, hsa-miR-566, hsa-miR-576-3p, hsa-miR-590-3p, hsa-miR-597, hsa-miR-601, hsa-miR-603, hsa-miR-604, hsa-miR-645, hsa-miR-660, hsa-miR-720, hsa-miR-875-5p, hsa-miR-886-3p, hsa-miR-886-5p, hsa-miR-1244, hsa-miR-1260, hsa-miR-1274A, hsa-miR-1275, hsa-miR-1276, and combinations thereof. In some embodiments, the disclosed methods may include detecting one or more nucleic acid molecules comprising a sequence of any of SEQ ID NOs:1-103 or combinations thereof.

The disclosed methods may include methods of assessing the likelihood of viability of the embryo based on the expression of one or more miRNAs by the embryo. In some embodiments, the detected miRNA in these methods is selected from a group consisting of hsa-mir-372, hsa-mir-645, hsa-mir-191, hsa-mir-376a, and hsa-mir-645. In particular, the detected miRNA in these methods may be selected from a group consisting of hsa-mir-372, hsa-mir-645, or both.

The disclosed methods may include methods of assessing the likelihood of euploidy or aneuploidy of the embryo based on the expression of one or more miRNAs by the embryo. In some embodiments, the detected miRNA in these methods is selected from a group consisting of hsa-mir-141, hsa-mir-1276, hsa-mir-27b, hsa-mir-518a-3p, hsa-mir-339-3p, hsa-mir-191, hsa-mir-30c, hsa-mir-29b, hsa-mir-192, and combinations thereof. In other embodiments of these methods, intracellular expression of a miRNA selected from a group consisting of hsa-mir-141, hsa-mir-1276, hsa-mir-27b, hsa-mir-518a-3p, hsa-mir-339-3p, and combinations thereof is detected. In further embodiments of these methods, extracellular expression of a miRNA selected from a group consisting of hsa-mir-191, hsa-mir-30c, hsa-mir-29b, hsa-mir-192, hsa-mir-27b, and combinations thereof is detected.

The disclosed methods may include methods of assessing the likelihood of whether the embryo is male or female. In some embodiments, the detected miRNA in these methods is selected from a group consisting of hsa-mir-206, hsa-mir-512-5p, hsa-mir-26b#, hsa-mir-518d-5p, hsa-mir-31, and combinations thereof.

The disclosed methods of assessment may be utilized in order to select an embryo as a suitable candidate for implantation into a patient. For example, the methods may include culturing an embryo in vitro (e.g., for a period of time that is sufficient for the embryo to develop into a blastocyst), detecting one or more miRNAs that are expressed by the embryo, and selecting the embryo for implantation based on the detected miRNAs. As such, the disclosed methods may include the additional step of implanting the selected embryo in the uterus of the patient. In some embodiments, the disclosed methods may be practiced by: (a) requesting a test providing results of an analysis to determine whether one or more miRNAs are expressed by a sample obtained from an embryo grown in vitro, which may include an intracellular sample or an extracellular sample such as culture media; and (b) selecting and implanting an embryo in the female patient based on the results of the test.

The disclosed methods may include methods of modifying culture media. For example, the methods of improving culture media may include supplementing or depleting the culture media of one or more miRNAs as disclosed herein. In some embodiments, the methods of improving culture media may include supplementing or depleting the culture media of one or more miRNAs in order to improve viability of the embryo, for example, with regard to subsequent implantation into a patient. The methods of improving culture media may include supplementing or depleting the culture media of one or more miRNAs prior to introducing the embryo to the culture media or during growth of the embryo in the culture media. In particular, hsa-mir-645 may be added to the culture media.

Also disclosed are kits that may be utilized to perform the disclosed methods. In some embodiments, the kits comprise, consist essentially of, or consist of oligonucleotide reagents for detecting each of hsa-mir-372, hsa-mir-645, hsa-mir-191, hsa-mir-376a, and hsa-mir-645 (e.g., primers and probes which may be labeled). In other embodiments, the kits comprise, consist essentially of or consist of oligonucleotide reagents for detecting each of hsa-mir-141, hsa-mir-1276, hsa-mir-27b, hsa-mir-518a-3p, hsa-mir-339-3p, hsa-mir-191, hsa-mir-30c, hsa-mir-29b, and hsa-mir-192 (e.g., primers and probes which may be labeled). In further embodiments, the kits comprise, consist essentially of, or consist of oligonucleotide reagents for detecting each of hsa-mir-206, hsa-mir-512-5p, hsa-mir-26b#, hsa-mir-518d-5p, and hsa-mir-31 (e.g., primers and probes which may be labeled). The kits may comprise, consist essentially of, or consist of additional reagents including enzymes for performing RT-PCR (e.g., a reverse transcriptase or a DNA polymerase, such as a thermostable polymerase) and/or buffers.

### BRIEF DESCRIPTION OF THE FIGURES

FIG. 1. Confirmed differential expression of miRNAs between euploid and aneuploid embryos by qPCR after normalization to the control probe, snRNA U6, which was consistently expressed in all samples. The vertical axis represents the number of fold changes of the miRNA between the two experimental groups. Error bars represent mean±s.e.m. and significant fold changes are marked by *p<0.01 and **p<0.05.

FIG. 2. Top 20 gene pathways targeted by miRNAs 27b, 141, 339-3p, and 345 predicted by DIANA Lab mirPath database and Targetscan 5 web-based software. Vertical bars represent genes targeted by each miRNA within known pathways as well the total number of genes targeted within each pathway.

FIG. 3. Implantation results for 55 single embryo transfer cases.

### DETAILED DESCRIPTION

The disclosed subject matter further may be described utilizing terms as defined below.

Unless otherwise specified or indicated by context, the terms “a”, “an”, and “the” mean “one or more.” For example, a “miRNA” should be interpreted to mean “one or more miRNAs.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus ≧10% of the particular term.

As used herein, the terms “include” and “including” have the same meaning as the terms “comprise” and “comprising.” For example, “a method that includes a step” should be interpreted to mean “a method that comprises a step.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and permitting the inclusion only of additional components that do not fundamentally alter the nature of the claimed subject matter.

As used herein, the term “detecting expression of an miRNA” means determining that the miRNA is being expressed or determining that the miRNA is not being expressed. In some embodiments, expression may be detected relative to expression of a control nucleic acid. Detecting expression of an miRNA may include detecting reduced expression of an miRNA relative to expression of a control nucleic acid, for example relative to expression of RNU48. Detecting expression of an miRNA may include detecting increased expression of an miRNA relative to expression of a control nucleic acid, for example relative to expression of RNA48.

As used herein, the term “modify” means changing a material from its initial state. For example, “modifying culture media” may include adding one or more miRNAs to the culture media or a component thereof or depleting one or more miRNAs from the culture media or a component thereof. Components of culture media may include a liquid growth media (e.g., classical media published by Dulbecco, Eagle, Ham, Moore, Morgan, and others) and a protein supplement. Accordingly, “modifying culture media” may include adding one or more miRNAs to a protein supplement or depleting one or more miRNAs from a protein supplement, wherein the protein supplement is added to a liquid growth media to form a culture media.

“MicroRNA” means an endogenous non-coding RNA between 18 and 25 nucleobases in length, which is the product of cleavage of a pre-miRNA by the enzyme Dicer. Examples of mature miRNAs are found in the miRNA database known as miRBase. In certain embodiments, microRNA is abbreviated as “miRNA” or “miR.”

“Pre-miRNA” or “pre-miR” means a non-coding RNA having a hairpin structure, which is the product of cleavage of a pri-miR by the double-stranded RNA-specific ribonuclease known as Drosha.

“Stem-loop sequence” means an RNA having a hairpin structure and containing a mature miRNA sequence. Pre-miRNA sequences and stem-loop sequences may overlap. Examples of stem-loop sequences are found in the miRNA database known as miRBase.

“Pri-miRNA” or “pri-miR” means a non-coding RNA having a hairpin structure that is a substrate for the double-stranded RNA-specific ribonuclease Drosha.

“miRNA precursor” means a transcript that originates from a genomic DNA and that comprises a non-coding, structured RNA comprising one or more miRNA sequences. For example, in certain embodiments a miRNA precursor is a pre-miRNA. In certain embodiments, a miRNA precursor is a pri-miRNA.

The presently disclosed methods may include detecting expression of miRNA. “Expression” means any functions and steps by which a gene's coded information is converted into structures present and operating in a cell. Detecting expression of miRNA may include detecting nucleic acid comprising miRNA, pre-miRNA, or pri-miRNA by suitable methods known in the art, including methods that include one or more of the following: reverse transcription, polymerase chain reaction, probing, targeting, and hybridization. MicroRNA expression may be assessed via detecting nucleic acid comprising miRNA, pre-miRNA, or pri-miRNA in an extracellular sample (e.g., in culture media in which an embryo has been grown) or in an intracellular sample (e.g., an intracellular sample of an embryo).

Detection methods may include hybridizing an oligonucleotide reagent to the miRNA and detecting hybridization of the primer or probe to the miRNA. Suitable oligonucleotide reagents may include primers and probes such as RNA probes and DNA probes, which optionally include a label (e.g., a radioisotope label, an enzymatic label, a fluorophore label, and the like).

Detection methods may include converting the miRNA to DNA via performing reverse transcription and amplifying the DNA via performing a polymerase chain reaction (RT-PCR). RNA linkers may be ligated to the miRNA prior to converting the miRNA to DNA and/or DNA linkers may be ligated to the DNA prior to amplifying the DNA. Multiple miRNAs may be detected in the methods (e.g., at least 2, 3, 4, 5, 6, 7, 8, 9, 10, 11, 12, 13, 14, 15, 16, 17, 18, 19, or 20 miRNAs) and microarrays comprising probes for multiple miRNAs may be utilized to detect multiple miRNAs.

“Target nucleic acid” means a nucleic acid to which an oligomeric compound is designed to hybridize. “Targeting” means the process of design and selection of nucleobase sequence that will hybridize to a target nucleic acid. “Targeted to” means having a nucleobase sequence that will allow hybridization to a target nucleic acid.

“Nucleobase sequence” means the order of contiguous nucleobases, in a 5′ to 3′ orientation, independent of any sugar, linkage, and/or nucleobase modification. “Contiguous nucleobases” means nucleobases immediately adjacent to each other in a nucleic acid. “Nucleobase complementarity” means the ability of two nucleobases to pair non-covalently via hydrogen bonding. “Complementary” means that an oligomeric compound is capable of hybridizing to a target nucleic acid under stringent hybridization conditions. “Fully complementary” means each nucleobase of an oligomeric compound is capable of pairing with a nucleobase at each corresponding position in a target nucleic acid. For example, in certain embodiments, an oligomeric compound wherein each nucleobase has complementarity to a nucleobase within a region of a miRNA stem-loop sequence is fully complementary to the miRNA stem-loop sequence. “Percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound. In certain embodiments, percent complementarity means the number of nucleobases that are complementary to the target nucleic acid, divided by the total number of nucleobases of the modified oligonucleotide. “Percent identity” means the number of nucleobases in first nucleic acid that are identical to nucleobases at corresponding positions in a second nucleic acid, divided by the total number of nucleobases in the first nucleic acid. “Hybridize” means the annealing of complementary nucleic acids that occurs through nucleobase complementarity. “Mismatch” means a nucleobase of a first nucleic acid that is not capable of pairing with a nucleobase at a corresponding position of a second nucleic acid. “Identical” means having the same nucleobase sequence.

The presently disclosed methods and kits may utilize or include oligonucleotide reagents for detecting miRNAs. As contemplated herein, “oligonucleotide reagents” may include oligonucleotides that hybridize specifically to a selected miRNA and that may be used to detect the miRNA based on the specific hybridization. For example, the oligonucleotide reagents may include one or more primers for performing any or all steps of RT-PCR performed on miRNA as contemplated herein (i.e., a primer for performing reverse transcription (RT) of an miRNA to obtain reverse transcribed miRNA and/or one or a pair of primers for performing polymerase chain reaction (PCR) of the reverse transcribed miRNA to obtain an amplified product). The reverse transcribed miRNA or amplified product then may be detected by methods known in the art. Oligonucleotide reagents may include probes for detecting an miRNA and/or any product of RT-PCR performed on miRNA (e.g., a probe for detecting a reverse transcribed miRNA, or a probe for detecting an amplified product of the reverse transcribed miRNA). Primers and probes as contemplated herein may include a label.

The disclosed methods may be utilized to detect expression of one or more miRNAs by an embryo or the lack of expression of one or more miRNAs by an embryo, for example in order to assess embryo viability or aneuploidy. The disclosed methods also may be utilized to optimize culture media for an embryo via adding one or more miRNAs to the culture media or depleting one or more miRNAs from the culture media.

Suitable miRNA's for the disclosed methods may include, but are not limited to, hsa-miR-17, hsa-miR-19, has-miR-19a, hsa-miR-19b, hsa-miR-20a, hsa-miR-24, hsa-miR-25, hsa-miR-26b, hsa-miR-26b#, hsa-miR-27b, hsa-miR-29b, hsa-miR-29b-1, hsa-miR-29b-2, hsa-miR-30a-5p, hsa-miR-30b, hsa-miR-30c, hsa-miR-30c-1, hsa-miR-30c-2, hsa-miR-31, hsa-miR-92a, hsa-miR-93, hsa-miR-106, hsa-miR-106a, hsa-miR-106b, hsa-miR-140-5p, hsa-miR-141, hsa-miR-146b-5p, has-miRNA-148a, hsa-miR-149, hsa-miR-151-3p, hsa-miR-151-5p, has-155, hsa-miR-182#, hsa-miR-191, hsa-miR-192, hsa-miR-193b, hsa-miR-200c, hsa-miR-206, hsa-miR-302a, hsa-miR-302a#, hsa-miR-302b, hsa-miR-302b#, hsa-miR-302c, hsa-miR-302d, hsa-miR-320, hsa-miR-339, hsa-miR-339-3p, hsa-miR-345, hsa-miR-346, hsa-miR-347, hsa-miR-362-3p, hsa-miR-367, hsa-miR-371-3p, hsa-miR-372, hsa-miR-373, hsa-miR-374a, hsa-miR-376a, hsa-miR-376a, hsa-miR-376a-1, hsa-miR-376a-2, hsa-miR-378, has-miR-380-5p, hsa-miR-454, hsa-miR-484, hsa-miR-487b, hsa-miR-494, hsa-miR-500, hsa-miR-509-5p, hsa-miR-512-1, hsa-miR-512-2, hsa-miR-512-3p, hsa-miR-512-5p, hsa-miR-517c, hsa-miR-518a-1, hsa-miR-518a-2, hsa-miR-518a-3p, hsa-miR-518c, hsa-miR-518d, hsa-miR-518d-5p, hsa-miR-511e, hsa-miR-519a, hsa-miR-520c-3p, hsa-miR-520D-3p, hsa-miR-520d-5p, hsa-miR-522, hsa-miR-525-3p, hsa-miR-548a-3p, hsa-miR-548c-3p, hsa-mir-548d-3p, hsa-miR-566, hsa-miR-576-3p, hsa-miR-590-3p, hsa-miR-597, hsa-miR-601, hsa-miR-603, hsa-miR-604, hsa-miR-645, hsa-miR-660, hsa-miR-720, hsa-miR-875-5p, hsa-miR-886-3p, hsa-miR-886-5p, hsa-miR-1244, hsa-miR-1260, hsa-miR-1274A, hsa-miR-1275, and hsa-miR-1276. (See Table 1.)

TABLE 1

| Assay Name | microRNA Sequence | SEQ ID NO | Accession Number |
|---|---|---|---|
| hsa-mR-17 | CAAAGUGCUUACAGUGCAGGUAG | 1 | MI0000071 |
| hsa-miR-19 | | | |
| hsa-miR-19a | UGUGCAAAUCUAUGCAAAACUGA | 2 | MI0000073 |
| hsa.miR-19b | UGUGCAAAUCCAUGCAAAACUGA | 3 | MI0000074/ |
| | | | MI0000075 |
| hsa-miR-20a | UAAAGUGCUUAUAGUGCAGGUAG | 4 | MI0000076 |
| hsa-miR-24 | UGGCUCAGUUCAGCAGGAACA | 5 | MI0000080/ |
| | | | MI0000081 |
| hsa-miR-25 | CAUUGCACUUGUCUCGGUCUGA | 6 | M10000082 |
| hsa-miR-26b | UUCAAGUAAUUCAGGAUAGGU | 7 | MI0000084 |
| hsa-m1R-26b# | CCUGUUCUCCAUUACUUGGCUC | 8 | MIMAT0004500 |
| hsa-miR-27b | UUCACAGUGGCUAAGUUCUGC | 9 | M10000440 |
| hsa-miR-29b | UAGCACCAUUUGAAAUCAGUGUU | 10 | MIMAT0000100 |
| hsa-miR-29b-1 | GCUCCUUUCAUAUGGUGGUUUAGA | 11 | MI0000105 |
| hsa-miR-29b-2 | CUGGUUUCACAUGGUGGCUUAG | 12 | MI0000107 |
| hsa-miR-30a-5p | UGUAAACAUCCUCGACUGGAAG | 13 | MIMAT0000087 |
| hsa-miR-30b | UGUAAACAUCCUACACUCAGCU | 14 | MI0000441 |
| hsa-miR-30c | UGUAAACAUCCUACACUCUCAGC | 15 | MIMAT0000244 |
| hsa-miR-30c-1 | CUGGGAGAGGGUUGUUUACUCC | 16 | MI0000736 |
| hsa-miR-30c-2 | CUGGGAGAAGGCUGUUUACUCU | 17 | MI0000254 |
| hsa-miR-31 | AGGCAAGAUGCUGGCAUAGCU | 18 | MI0000089 |
| hsa-miR-92a | UAUUGCACUUGUCCCGGCCUGU | 19 | MI0000093/ |
| | | | MI0000094 |
| hsa-miR-93 | AAAGUGCUGUUCGUGCAGGUAG | 20 | MI0000095 |
| hsa-miR-106 | | | |
| hsa-miR-106a | AAAAGUGCUUACAGUGCAGGUAG | 21 | MI0000103 |
| hsa-miR-106b | UAAAGUGCUGACAGUGCAGAU | 22 | MI0000734 |
| hsa-miR-140-5p | CAGUGGUUUUACCCUAUGGUAG | 23 | MIMAT0000431 |
| hsa-miR-141 | UAACACUGUCUGGUAAAGAUGG | 24 | MI0000457 |
| hsa-miR-146b-5p | UGAGAACUGAAUUCCAUAGGCU | 25 | MIMAT0002809 |
| hsa-miR-148a | UCAGUGCACUACAGAACUUUGU | 26 | MI0000253 |
| hsa-miR-149 | UCUGCUCCGUGUCUUCACUCCC | 27 | MI0000478 |
| hsa-miR-151-3p | CUAGACUGAAGCUCCUUGAGG | 28 | MIMAT0000757 |
| hsa-miR-151-5p | UCGAGGAGCUCACAGUCUAGU | 29 | MIMAT004697 |
| hsa-155 | UUAAUGCUAAUCGUGAUAGGGGU | 30 | MI0000681 |
| hsa-miR-182# | UGGUUCUAGACUUGCCAACUA | 31 | MIMAT0000260 |
| hsa-miR-191 | CAACGGAAUCCCCAAAAGCAGCUG | 32 | MI0000465 |
| hsa-miR-192 | CUGACCUAUGAAUUGACAGCC | 33 | MI0000234 |
| hsa-mIR-193b | AACUGGCCCUCAAAGUCCCGCU | 34 | MI0003137 |
| hsa-miR-200c | UAAUACUGCCGGGUAAUGAUGGGA | 35 | MI0000650 |
| hsa-miR-206 | UGGAAUGUAAGGAAGUGUGUGG | 36 | MI0000490 |
| hsa-miR-302a | UAAGUGCUUCCAUGUUUUGGUGA | 37 | MI0000738 |
| hsa-miR-302a# | ACUUAAACGUGGAUGUACUUGCU | 38 | MIMAT0000683 |
| hsa-miR-302b | UAAGUGCUUCCAUGUUUUAGUAG | 39 | MI0000772 |
| hsa-miR-302b# | ACUUUAACAUGGAAGUGCUUUC | 40 | MIMAT0000714 |
| hsa-miR-302c | UAAGUGCUUCCAUGUUUCAGUGG | 41 | MI0000773 |
| hsa-miR-302d | UAAGUGCUUCCAUGUUUGAGUGU | 42 | MI0000774 |
| hsa-miR-320 | AAAAGCUGGGUUGAGAGGGCGA | 43 | MI0000542 |
| hsa-miR-339 | UCCCUGUCCUCCAGGAGCUCA | 44 | MI0000815 |
| hsa-miR-339-3p | UGAGCGCCUCGACGACAGAGCCG | 45 | MIMAT0004702 |
| hsa-miR-345 | GCUGACUCCUAGUCCAGGGCUC | 46 | MI0000825 |
| hsa-miR-346 | UGUCUGCCCGCAUGCCUGCCUCU | 47 | MI0000826 |
| hsa-miR-347 | | | |
| hsa-miR-362-3p | AACACACCUAUUCAAGGAUUCA | 48 | MIMAT0004683 |
| hsa-miR-367 | AAUUGCACUUUAGCAAUGGUGA | 49 | MI0000775 |
| hsa-miR-371-3p | AAGUGCCGCCAUCUUUUGAGUGU | 50 | MIMAT0000723 |
| hsa-miR-372 | AAAGUGCUGCGACAUUUGAGCGU | 51 | MI0000780 |
| hsa-miR-373 | GAAGUGCUUCGAUUUUGGGGUGU | 52 | MI0000781 |
| hsa-miR-374a | UUAUAAUACAACCUGAUAAGUG | 53 | MI0000782 |
| hsa-miR-376a | AUCAUAGAGGAAAAUCCACGU | 54 | MI0000784/ |
| | | | MI0003529 |
| hsa-miR-376a-1 | AUCAUAGAGGAAAUCCAUGUU | 55 | MI0000784 |
| hsa-miR-376a-2 | UGGUGGGCCGCAGAACAUGUGC | 56 | MI0003529 |
| hsa-miR-378 | CUCCUGACUCCAGGUCCUGUGU | 57 | MI0000786 |
| hsa-miR-380-5p | UGGUUGACCAUAGAACAUGCGC | 58 | MIMAT0000734 |
| hsa-miR-454 | UAGUGCAAUAUUGCUUAUAGGGU | 59 | MI0003820 |
| hsa-miR-484 | UCAGGCUCAGUCCCCUCCGAU | 60 | MI0002468 |
| hsa-miR-487b | AAUCGUACAGGGUCAUCCACUU | 61 | MI0003530 |
| hsa-miR-494 | UGAAACAUACACGGGAAACCUC | 62 | MI0003134 |
| hsa-miR-500 | UAAUCCUUGCUACCUGGGUGAGA | 63 | MI0003184 |
| hsa-miR-509-5p | UACUGCAGACAGUGGCAAUCA | 64 | MIMAT0004779 |
| hsa-miR-512-1 | AAGUGCUGUCAUAGCUGAGGUC | 65 | MI0003140 |
| hsa-miR-512-2 | CACUCAGCCUUGAGGGCACUUUC | 66 | MI0003141 |
| hsa-miR-512-3p | AAGUGCUGUCAUAGCUGAGGUC | 67 | MIMAT0002823 |
| hsa-miR-512-5p | CACUCAGCCUUGAGGGCACUUUC | 68 | MIMAT0002822 |
| hsa-miR-517c | AUCGUGCAUCCUUUUAGAGUGU | 69 | MI0003174 |
| hsa-miR-518a-1 | CUGCAAAGGGAAGCCCUUUC | 70 | MI0003170 |
| hsa-miR-518a-2 | GAAAGCGCUUCCCUUUGCUGGA | 71 | MI0003173 |
| hsa-m1R-518a-3p | GAAAGCGCUUCCCUUUGCUGGA | 72 | MIMAT0002863 |
| hsa-miR-518c | CAAAGCGCUUCUCUUUAGAGUGU | 73 | MI0003159 |
| hsa-miR-518d | CAAAGCGCUUCCCUUUGGAGC | 74 | MI0003171 |
| hsa-miR-518d-5p | CUCUAGAGGGAAGCACUUUCUG | 75 | MIMAT0005456 |
| hsa-miR-518e | AAAGCGCUUCCCUUCAGAGUG | 76 | MI0003169 |
| hsa-mIR-519a | AAAGUGCAUCCUUUUAGAGUGU | 77 | MI0003178/ |
| | | | MI0003182 |
| hsa-miR-520c-3p | AAAGUGCUUCCUUUUAGAGGGU | 78 | MIMAT0002846 |
| hsa-miR-520c-3p | AAAGUGCUUCCUUUUAGAGGGU | 79 | MIMAT0002856 |
| hsa-miR-520d-5p | CUACAAAGGGAAGCCCCUUUC | 80 | MIMAT0002855 |
| hsa-miR-522 | AAAAUGGUUCCCUUUAGAGUGU | 81 | MI0003177 |
| hsa-miR-525-3p | GAAGGCGCUUCCCUUUAGAGCG | 82 | MIMAT0002839 |
| hSa-miR-548a-3p | CAAAACUGGCAAUUACUUUUGC | 83 | MIMAT0003251 |
| hsa-miR-548c-3p | CAAAAAUCUCAAUUACUUUUGC | 84 | MIMAT0003285 |
| hsa-miR-548d-3p | CAAAAACCACAGUUUCUUUUGC | 85 | MIMAT0003323 |
| hsa-miR-566 | GGGCGCCUGUGAUCCCAAC | 86 | MI0003572 |
| hsa-miR-576-3p | AAGAUGUGGAAAAAUUGGAAUC | 87 | MIMAT0004796 |
| hsa-miR-590-3p | UAAUUUUAUGUAUAAGCUAGU | 88 | MIMAT0004801 |
| hsa-miR-597 | UGUGUCACUCGAUGACCACUGU | 89 | MI0003609 |
| hsa-miR-601 | UGGUCUAGGAUUGUUGGAGGAG | 90 | MI0003614 |
| hsa-miR-603 | CACACACUGCAAUUACUUUUGC | 91 | MI0003616 |
| hsa-miR-604 | AGGCUGCGGAAUUCAGGAC | 92 | MI0003617 |
| hsa-miR-645 | UCUAGGCUGGUACUGCUGA | 93 | MI0003660 |
| hsa-miR-660 | UACCCAUUGCAUAUCGGAGUUG | 94 | MI0003684 |
| hsa-miR-720 | UCUCGCUGGGGCCUCCA | 95 | MI0006654 |
| hsa-miR-875-5p | UAUACCUCAGUUUUAUCAGGUG | 96 | MIMAT0004922 |
| hsa-miR-886-3p | CGCGGGUGCUUACUGACCCUU | 97 | |
| hsa-miR-886-5p | CGGGUCGGAGUUAGCUCAAGCGG | 98 | MIMAT0004905 |
| hsa-miR-1244 | AAGUAGUUGGUUUGUAUGAGAUGGUU | 99 | MI0006379/ |
| | | | MI0015974/ |
| | | | MI0015975 |
| hsa-miR-1260 | AUCCCACCUCUGCCACCA | 100 | MI0006394 |
| hsa-miR-1274A | GUCCCUGUUCAGGCGCCA | 101 | MIMAT0005927 |
| hsa-miR-1275 | GUGGGGGAGAGGCUGUC | 102 | MI0006415 |
| hsa-miR-1276 | UAAAGAGCCCUGUGGAGACA | 103 | MI0006416 |

As used herein, the term “patient,” which may be used interchangeably with the terms “subject” or “individual,” refers to one who receives medical care, attention or treatment and may encompass a human patient. The disclosed methods may include selecting an embryo for implantation based on detecting expression of one or more miRNAs, and subsequently implanting the embryo in a patient. As such, the term “patient” is meant to encompass a person that has elected to undergo implantation of an embryo into the patient's uterus. In some embodiments, the methods may include requesting a test that provides the results of an analysis of expression of one or more miRNAs in a sample obtained from an embryo grown in vitro (e.g., and intracellular sample or an extracellular sample such as culture media in which the embryo has been grown); selecting an embryo for implantation in a patient based on the results of the test; and optionally, implanting the selected embryo in a patient's uterus.

The embryo utilized in the disclosed methods may be an embryo obtained by fertilizing an oocyte from a patient that has elected to undergo embryo transfer (e.g., after explanting the oocyte from the patient and performing in vitro fertilization (IVT) on the oocyte, for example, by intracytoplasmic sperm injection (ISCI)). Alternatively, the embryo utilized in the disclosed methods may be an embryo obtained by fertilizing an oocyte from a donor, for example, where: 1) the patient has undergone ovarian failure, e.g., ovarian failure caused by radiation therapy, chemotherapy, surgical removal of the ovaries and a variety of disease states which cause or are associated with ovarian failure; 2) the patient carries a genetic disease which the patient does not want passed on to their offspring; 3) that patient is a woman whose age is sufficiently advanced so that their fertility potential is impaired significantly; and 4) the patient has provided poor quality embryos during previous IVF cycles.

### ILLUSTRATIVE EMBODIMENTS

The following examples are illustrative and are not intended to limit the disclosed subject matter.

### Embodiment 1

A method for selecting an embryo that has been grown in culture media in vitro for implantation into a patient, the method comprising detecting expression of one or more miRNAs by the embryo.

### Embodiment 2

The method of embodiment 1, comprising detecting intracellular expression.

### Embodiment 3

The method of any of the foregoing embodiments, comprising detecting extracellular expression.

### Embodiment 4

The method of any of the foregoing embodiments, wherein detecting expression of the one or more miRNAs by the embryo comprises contacting a sample of the culture media with a reagent that detects the one or more miRNAs.

### Embodiment 5

The method of embodiment 4, wherein the reagent is an oligonucleotide that hybridizes to the one or more miRNAs.

### Embodiment 6

The method of embodiment 5, wherein the oligonucleotide is DNA.

### Embodiment 7

The method of any of the foregoing embodiments, wherein detecting comprises converting the one or more miRNAs to DNA via performing reverse transcription and amplifying the DNA via performing a polymerase chain reaction.

### Embodiment 8

The method of any of the foregoing embodiments, wherein the embryo has been grown in the culture media in vitro for at least about 3 days.

### Embodiment 9

The method of embodiment 8, wherein the embryo has developed into a blastocyst.

### Embodiment 10

The method of any of the foregoing embodiments, wherein the embryo is formed from an oocyte that was fertilized via in vitro fertilization

### Embodiment 11

The method of embodiment 10, wherein the in vitro fertilization includes intracytoplasmic sperm injection (ICSI).

### Embodiment 12

The method of any of the foregoing embodiments, wherein the miRNA is selected from a group consisting of hsa-miR-17, hsa-miR-19, has-miR-19a, hsa-miR-19b, hsa-miR-20a, hsa-miR-24, hsa-miR-25, hsa-miR-26b, hsa-miR-26b#, hsa-miR-27b, hsa-miR-29b, hsa-miR-29b-1, hsa-miR-29b-2, hsa-miR-30a-5p, hsa-miR-30b, hsa-miR-30c, hsa-miR-30c-1, hsa-miR-30c-2, hsa-miR-31, hsa-miR-92a, hsa-miR-93, hsa-miR-106, hsa-miR-106a, hsa-miR-106b, hsa-miR-140-5p, hsa-miR-141, hsa-miR-146b-5p, has-miRNA-148a, hsa-miR-149, hsa-miR-151-3p, hsa-miR-151-5p, has-155, hsa-miR-182#, hsa-miR-191, hsa-miR-192, hsa-miR-193b, hsa-miR-200c, hsa-miR-206, hsa-miR-302a, hsa-miR-302a#, hsa-miR-302b, hsa-miR-302b#, hsa-miR-302c, hsa-miR-302d, hsa-miR-320, hsa-miR-339, hsa-miR-339-3p, hsa-miR-345, hsa-miR-346, hsa-miR-347, hsa-miR-362-3p, hsa-miR-367, hsa-miR-371-3p, hsa-miR-372, hsa-miR-373, hsa-miR-374a, hsa-miR-376a, hsa-miR-376a, hsa-miR-376a-1, hsa-miR-376a-2, hsa-miR-378, has-miR-380-5p, hsa-miR-454, hsa-miR-484, hsa-miR-487b, hsa-miR-494, hsa-miR-500, hsa-miR-509-5p, hsa-miR-512-1, hsa-miR-512-2, hsa-miR-512-3p, hsa-miR-512-5p, hsa-miR-517c, hsa-miR-518a-1, hsa-miR-518a-2, hsa-miR-518a-3p, hsa-miR-518c, hsa-miR-518d, hsa-miR-518d-5p, hsa-miR-518e, hsa-miR-519a, hsa-miR-520c-3p, hsa-miR-520D-3p, hsa-miR-520d-5p, hsa-miR-522, hsa-miR-525-3p, hsa-miR-548a-3p, hsa-miR-548c-3p, hsa-mir-548d-3p, hsa-miR-566, hsa-miR-576-3p, hsa-miR-590-3p, hsa-miR-597, hsa-miR-601, hsa-miR-603, hsa-miR-604, hsa-miR-645, hsa-miR-660, hsa-miR-720, hsa-miR-875-5p, hsa-miR-886-3p, hsa-miR-886-5p, hsa-miR-1244, hsa-miR-1260, hsa-miR-1274A, hsa-miR-1275, and hsa-miR-1276.

### Embodiment 13

The method of any of the foregoing embodiments, wherein the one or more detected miRNAs indicate whether the embryo is viable.

### Embodiment 14

The method of embodiment 13, wherein the miRNA is selected from a group consisting of hsa-mir-372, hsa-mir-645, hsa-mir-191, hsa-mir-376a, and hsa-mir-645.

### Embodiment 15

The method of embodiment 13, comprising detecting extracellular expression of hsa-mir-372 or hsa-mir-645.

### Embodiment 16

The method of embodiment 13, comprising detecting extracellular expression of hsa-mir-372 and hsa-mir-645.

### Embodiment 17

The method of any of the foregoing embodiments, wherein the one or more detected miRNAs indicate whether the embryo is euploid or aneuploid.

### Embodiment 18

The method of embodiment 17, wherein the miRNA is selected from a group consisting of hsa-mir-141, hsa-mir-1276, hsa-mir-27b, hsa-mir-518a-3p, hsa-mir-339-3p, hsa-mir-191, hsa-mir-30c, hsa-mir-29b, and hsa-mir-192.

### Embodiment 19

The method of embodiment 17, comprising detecting intracellular expression of a miRNA selected from a group consisting of hsa-mir-141, hsa-mir-1276, hsa-mir-27b, hsa-mir-518a-3p, and hsa-mir-339-3p.

### Embodiment 20

The method of embodiment 17, comprising detecting extracellular expression of a miRNA selected from a group consisting of hsa-mir-191, hsa-mir-30c, hsa-mir-29b, hsa-mir-192, and hsa-mir-27b.

### Embodiment 21

The method of any of the foregoing embodiments, wherein the one or more detected miRNAs indicate whether the embryo is male or female.

### Embodiment 22

The method of embodiment 21, wherein the miRNA is selected from a group consisting of hsa-mir-206, hsa-mir-512-5p, hsa-mir-26b#, hsa-mir-518d-5p, and hsa-mir-31.

### Embodiment 23

The method of any of the foregoing embodiments, further comprising implanting the selected embryo in the uterus of the patient.

### Embodiment 24

A method for improving viability of an embryo grown in culture media in vitro, the method comprising supplementing or depleting the culture media of one or more miRNAs and growing the embryo in the culture media.

### Embodiment 25

The method of embodiment 24, wherein the culture media is supplemented or depleted of one or more miRNAs prior to introducing the embryo to the culture media.

### Embodiment 26

The method of embodiment 24 or 25, wherein the culture media is supplemented or depleted of one or more miRNAs while the embryo is growing in the culture media.

### Embodiment 27

The method of any of embodiments 24-26, comprising supplementing the culture media with hsa-mir-645.

### Embodiment 28

The method of any of embodiments 24-27, wherein the one or more miRNAs are selected from a group consisting of hsa-miR-17, hsa-miR-19, has-miR-19a, hsa-miR-19b, hsa-miR-20a, hsa-miR-24, hsa-miR-25, hsa-miR-26b, hsa-miR-26b#, hsa-miR-27b, hsa-miR-29b, hsa-miR-29b-L, hsa-miR-29b-2, hsa-miR-30a-5p, hsa-miR-30b, hsa-miR-30c, hsa-miR-30c-1, hsa-miR-30c-2, hsa-miR-31, hsa-miR-92a, hsa-miR-93, hsa-miR-106, hsa-miR-106a, hsa-miR-106b, hsa-miR-140-5p, hsa-miR-141, hsa-miR-146b-5p, has-miRNA-148a, hsa-miR-149, hsa-miR-151-3p, hsa-miR-151-5p, has-155, hsa-miR-182#, hsa-miR-191, hsa-miR-192, hsa-miR-193b, hsa-miR-200c, hsa-miR-206, hsa-miR-302a, hsa-miR-302a#, hsa-miR-302b, hsa-miR-302b#, hsa-miR-302c, hsa-miR-302d, hsa-miR-320, hsa-miR-339, hsa-miR-339-3p, hsa-miR-345, hsa-miR-346, hsa-miR-347, hsa-miR-362-3p, hsa-miR-367, hsa-miR-371-3p, hsa-miR-372, hsa-miR-373, hsa-miR-374a, hsa-miR-376a, hsa-miR-376a, hsa-miR-376a-1, hsa-miR-376a-2, hsa-miR-378, has-miR-380-5p, hsa-miR-454, hsa-miR-484, hsa-miR-487b, hsa-miR-494, hsa-miR-500, hsa-miR-509-5p, hsa-miR-512-1, hsa-miR-512-2, hsa-miR-512-3p, hsa-miR-512-5p, hsa-miR-517c, hsa-miR-518a-1, hsa-miR-518a-2, hsa-miR-518a-3p, hsa-miR-518c, hsa-miR-518d, hsa-miR-518d-5p, hsa-miR-518e, hsa-miR-519a, hsa-miR-520c-3p, hsa-miR-520D-3p, hsa-miR-520d-5p, hsa-miR-522, hsa-miR-525-3p, hsa-miR-548a-3p, hsa-miR-548c-3p, hsa-mir-548d-3p, hsa-miR-566, hsa-miR-576-3p, hsa-miR-590-3p, hsa-miR-597, hsa-miR-601, hsa-miR-603, hsa-miR-604, hsa-miR-645, hsa-miR-660, hsa-miR-720, hsa-miR-875-5p, hsa-miR-886-3p, hsa-miR-886-5p, hsa-miR-1244, hsa-miR-1260, hsa-miR-1274A, hsa-miR-1275, and hsa-miR-1276.

### Embodiment 29

The method of any of embodiments 24-28, further comprising implanting the embryo in the uterus of a patient.

### Embodiment 30

A kit comprising, consisting essentially of, or consisting of oligonucleotide reagents for detecting each of hsa-mir-372, hsa-mir-645, hsa-mir-191, hsa-mir-376a, and hsa-mir-645.

### Embodiment 31

The kit of embodiment 30 further comprising, consisting essentially of, or consisting of one or more enzymes for performing any step of RT-PCR.

### Embodiment 32

A kit comprising, consisting essentially of, or consisting of oligonucleotide reagents for detecting each of hsa-mir-141, hsa-mir-1276, hsa-mir-27b, hsa-mir-518a-3p, hsa-mir-339-3p, hsa-mir-191, hsa-mir-30c, hsa-mir-29b, and hsa-mir-192.

### Embodiment 33

The kit of embodiment 32 further comprising, consisting essentially of, or consisting of one or more enzymes for performing any step of RT-PCR.

### Embodiment 34

A kit comprising, consisting essentially of, or consisting of oligonucleotide reagents for detecting each of hsa-mir-206, hsa-mir-512-5p, hsa-mir-26b#, hsa-mir-518d-5p, and hsa-mir-31.

### Embodiment 35

The kit of embodiment 34 further comprising, consisting essentially of, or consisting of one or more enzymes for performing any step of RT-PCR.

### EXAMPLES

The following examples are illustrative and are not intended to limit the disclosed subject matter.

### Example I

### Analysis of Embryonic miRNA Expression

Materials and Methods

Embryo Culture.

Under a University of Iowa institutional review board-approved protocol, couples whom previously undergone IVF donated their excess embryos for research. Embryos were cryopreserved at the pronuclear stage (day 1 embryos). In order to eliminate the possibility of sperm contamination, only intracytoplasmic sperm injection (ICSI) cycles were used.

Embryos were thawed and cultured in microdrops under light mineral oil in an environment of 5% to 6% CO₂ in air at 37° C. as follows: day 1 embryos were cultured in groups of 4 in 50-μL drops of IVC-One medium (InVitroCare, Inc) supplemented with 20% serum protein substitute (Cooper Surgical, Inc.) for 72 hours. Day 4 embryos were transferred to individual 8-μL drops of IVC-Three (InVitroCare, Inc.) and 20% serum protein substitute (Cooper Surgical, Inc.) for 24 hours. Before moving embryos to fresh drops, they were rinsed through five wash drops to eliminate transfer of spent media into the fresh drops. Six μl of embryo-conditioned (spent) media from individual culture drops were collected on day 5 and were immediately placed into 6 μl of lysis buffer with DNase (TaqMan Micro RNA Cells-to-CT Kit, Applied Biosystems, Inc.). After 8 minutes stop solution was added and the samples were stored at −80° C.

All embryos were cultured in sequential media to the blastocyst stage. On day 5 of embryonic development a 10-μm channel was opened in the zona pellucida with a series of 3-5 laser pulses at 5 milliseconds duration. (Octax Microscience, GmbH). A trophectoderm (TE) biopsy was performed on day 5 to 6 of embryo culture. Herniating TE cells were aspirated into a biopsy pipette and detached from the blastocyst by firing several pulses at the area of constriction. After several passes through wash solution, the biopsied cells were placed into a polymerase chain reaction tube with lysis buffer supplied by the Genesis Genetics Institute. The biopsies then were shipped on dry ice to Genesis Genetics Institute for comparative genomic hybridization. After TE biopsy the embryos were placed into 7 μl of lysis buffer with DNase (TaqMan Micro RNA Cells-to-CT Kit, Applied Biosystems, Inc.) After 8 minutes stop solution was added and the samples were stored at −80° C.

Under an additional University of Iowa institutional review board approved protocol patients undergoing in vitro fertilization were consented. The spent culture media samples surrounding individual embryos were collected from consented individuals undergoing single embryo transfer. The embryo culture conditions were identical to the above protocol with the exception that the culture drop sizes were 15 μl. Twelve μl of media were collected on day 5 of culture and stored at −80° C. Six μl were then placed in an equal volume of lysis solution and processed as above. Pregnancy outcomes from these individuals were gathered and used for analysis.

miRNA RT.

In order to maximize the total amount of RNA available, the direct Cells-to-Ct method was used for reverse transcription. A total of 3 μl of sample in Cells-to-Ct lysis buffer was used in combination with the human A and B Megaplex RT primer pools (Applied Biosystems, Inc.) and Taqman MicronRNA Reverse transcription Kit components (Applied Biosystems, Inc.) to allow the simultaneous reverse transcription of 754 human miRNA, 3 endogenous miRNA controls, and non-human negative controls. Total volume was 7.5 μl. The thermal-cycling conditions were as follow

| Stage | Temp | Time |
|---|---|---|
| Cycle | 16° C. | 2 min |
| (40 Cycles) | 42° C. | 1 min |
| | 50° C. | 1 sec |
| Hold | 65° C. | 5 min |
| Hold | 4° C. | ∞ |

Pre-Amplification.

2.5 μl of Megaplex RT product was then pre-amplified using TaqMan PreAmp Master Mix (2×) and Megaplex PreAmp Primers (10×) (Applied Biosystems, Inc.). The final pre-amplified product was not diluted. The total reaction volume was 25 μl and the thermal-cycling conditions were as follows:

| Stage | Temp | | Time | |
|---|---|---|---|---|
| Hold | 95° | C. | 10 | min |
| Hold | 55° | C. | 2 | min |
| Hold | 72° | C. | 2 | min |
| Cycle | 95° | C. | 15 | sec |
| (12 Cycles) | 60° | C. | 4 | min |
| Hold^{‡} | 99.9° | C. | 10 | min |

| | | | |
|---|---|---|---|
| Hold | 4° | C. | ∞ |

PCR.

Individual pre-amplified RT products from embryos were profiled using two (A+B) 384-well TaqMan Low Density Array microfluidic cards with a final dilution of 1:100. RT products from spent media samples were profiled similarly with the exception of a final dilution of 1:16 (Applied Biosystems, Inc.). The arrays were loaded per the manufacturer's instructions. The PCR was performed on a 7900HT Fast Real-Time PCR System (Applied Biosystems, Inc.). The real-time data were analyzed by using SDS RQ manager and DataAssist software (Applied Biosystems, Inc.). The expression data was normalized to the top 2 least variant miRNA across all samples and with the DataAssist software global normalization tool. Top differentially expressed (p<0.05) miRNAs meeting all three normalization conditions were chosen for confirmation. Candidate miRNAs identified by array data were confirmed by using individual miRNA primers specific for RT and real-time reactions following the protocol of Taqman miRNA assays with pre-amplification (Applied Biosystems, Inc.). Individual miRNA assay PCR samples were performed in triplicate with no-template controls.

Results

Differential Expression of miRNAs in Human Euploid Versus Aneuploid Embryos.

The top 5 differentially expressed miRNAs in human euploid versus aneuploid embryos were identified and are listed in Table 2 (p<0.05).

| miRNA | Fold Change |
|---|---|
| hsa-mir-141 | 87 |
| hsa-mir-1276 | 34 |
| hsa-mir-27b | 28 |
| hsa-mir-518a-3p | 21 |
| hsa-mir-339-3p | 19 |

Differential Expression of miRNAs in Human Male Versus Female Embryos.

The top 5 differentially expressed miRNAs in human male versus female embryos were identified and are listed in Table 3 (p<0.05).

TABLE 3

| miRNA | Fold Change |
|---|---|
| hsa-mir-206 | −97 |
| hsa-mir-512-5p | 70 |
| hsa-mir-26b# | 54 |
| hsa-mir-518d-5p | 42 |
| hsa-mir-31 | 10 |

Differential Expression of miRNAs in Embryonic Culture Media from Euploid Versus Aneuploid Embryos.

The top 5 differentially expressed miRNAs in culture media from euploid versus aneuploid embryos were identified and are listed in Table 4 (p<0.01).

TABLE 4

| miRNA | Fold Change |
|---|---|
| hsa-mir-191 | −65 |
| hsa-mir-30c | 48 |
| hsa-mir-29b | 14 |
| hsa-mir-192 | 5 |
| hsa-mir-27b | 3 |

Identification of miRNA not Present in Culture Media Prior to Exposure to Embryos.

Four (4) miRNAs were identified that were not present in culture media prior to exposure to embryos. These included: hsa-mir-372, hsa-mir-191, hsa-mir-345, and hsa-mir-376a.

Identification of miRNA Present in Culture Media Prior to Exposure to Embryos but not Detectable after Exposure to Embryos.

One miRNA was identified (hsa-mir-645) that was present in culture media prior to exposure to embryos but not detectable after exposure to embryos.

Analysis of Media from Single Embryo Transfer Patients.

Embryos were grown in media and single embryos were transferred into patients. It was observed that hsa-mir-372 was 13-fold higher expressed in the media of embryos with cardiac activity versus media of embryos that either failed to implant or died before cardiac activity began. It was also observed that by testing for mir-372 and mir-645 there was a likelihood of 75% that an embryo would not implant if both tests were negative and a likelihood of 87% that an embryo would implant if both tests were positive.

### Example II

### MicroRNA Expression in the Human Blastocyst

Reference is made to Rosenbluth, et al. (Fertil. Steril. 2013 Mar. 1; 99(3):855-861. Epub 2012 Dec. 1), the content of which is incorporated herein by reference in its entirety.

Abstract

Objective:

To determine the most highly expressed microRNAs (miRNAs) in human blastocysts and to compare miRNAs in euploid versus aneuploid embryos and in male versus female embryos.

Design:

Experimental study of human embryos-14 blastocysts (four male, five female, and five aneuploid) were evaluated for miRNA expression using an array-based quantitative real time PCR (qPCR). Highly expressed and differentially expressed miRNAs were confirmed using qPCR in an expanded set of 28 blastocysts (eight male, eleven female, and nine aneuploid).

Setting:

Academic In Vitro Fertilization (IVF) program.

Outcome Measures:

Relative miRNA expression in individual blastocysts.

Results:

The most highly expressed miRNA in euploid embryos was miR-372. Many of the highly expressed miRNAs have been shown to be critical to mammalian embryo development and to maintenance of stem cell pluripotency. Several differentially expressed miRNAs were discovered based on chromosomal makeup, including sex of the embryo.

Conclusions:

Human blastocysts express miRNAs, which may be important to their survival. Differential miRNA expression based on sex implies some degree of differentiation at the blastocyst stage of development. Differential miRNA expression between euploid and aneuploid embryos may be an early indicator of their prognosis or a mechanism behind their eventual fate.

Introduction

MicroRNAs (miRNAs) are small (approximately 22 nucleotides) noncoding RNAs that regulate gene expression and have been implicated in a wide array of biologic processes including early embryo development and stem cell differentiation (1). The importance of miRNAs in early embryo development has been demonstrated in many species from C elegans to mammals (1-4). Targeted deletion in mice of the endonuclease Dicer1, which is critical for mature miRNAs biogenesis, has shown to arrest embryo development on day 6.5 to 7.5 of its 21-day gestational period (5). Recent experiments targeting specific miRNA gene clusters have identified families of miRNAs that are critical to mouse embryo and germ cell development (6, 7).

Despite their proven importance in embryo development, little is known about miRNA production in human embryos. Our objective was to first characterize the most abundant miRNAs produced in human blastocysts and compare these findings to those in lower species. Second, we sought to compare miRNA abundance in euploid versus aneuploid embryos to discover any differences that might lead to changes in embryo development. Finally, we compared male versus female embryos in terms of miRNA expression at the blastocyst stage.

Materials and Methods

Embryo Culture.

The study design was to screen an initial set of cultured embryos for relative and differential miRNA expression using an array-based quantitative real time PCR (qPCR) method. To confirm the miRNA array findings, an expanded set of embryos was tested for miRNA expression with single miRNA qPCR assays. All experiments were performed under a University of Iowa Institutional Review Board (IRB) approved protocol.

Excess cryopreserved embryos from IVF cycles were donated for scientific research. Patients utilized IVF for a variety of infertility diagnoses (undisclosed due to IRB constraints) and had oocytes inseminated by intracytoplasmic sperm injection (ICSI). Patients utilizing standard IVF were excluded to prevent sample contamination by accessory sperm. Pronuclear stage embryos were cryopreserved by controlled rate freezing 18 to 22 hours post-ICSI in 1.5 M 1,2 propanediol (PROH; Sigma, St. Louis, Mo.) as previously described (8). Embryos were thawed by air warming for 40 seconds followed by 10 second exposure to 30° C. sterile water. Cryoprotectants were removed in a stepwise dilutional fashion.

Surviving embryos were cultured in groups of three to four in 50 μl microdrops of IVC-One (In VitroCare; Frederick, Md.) supplemented with 20% SPS (Serum Protein Substitute, CooperSurgical Inc., Sage, Pasadena, Calif.) under oil (Cook Medical, Bloomington, Ind.) in 5.5-6.0% CO2 in air at 37° C. Embryos were moved to fresh drops of IVC-One supplemented with 20% SPS on day three. On the morning of day four, embryos were moved to individual culture in 8 μl of IVC-Three (In VitroCare) supplemented with 20% SPS. Before moving embryos to fresh drops, they were rinsed through a series of five wash drops. All embryos were cultured to the blastocyst stage and were graded according a standardized classification system (9).

Embryo Biopsy and Determination of Chromosomal Makeup.

Embryos were graded on day five of culture. Embryos that had reached at least the early blastocyst stage and had an inner cell mass grade of at least a B were chosen for assisted hatching. In these blastocysts a 10-μm channel was opened in the zona pellucida with a series of three to five laser pulses of 5 milliseconds duration (Octax Microscience, GmbH). On day five to six of culture, approximately five herniating trophectoderm (TE) cells per embryo were aspirated into a biopsy pipette and detached by firing several pulses at the area of constriction. After several passes through a wash solution, the biopsied cells were placed into a polymerase chain reaction tube with lysis buffer supplied by the Genesis Genetics Institute. The biopsies then were shipped on dry ice to Genesis Genetics Institute for array comparative genomic hybridization (aCGH) by their standard proprietary diagnostic technique.

miRNA Isolation and Reverse Transcription.

In order to maximize the total amount of RNA available, the direct Cells-to-Ct method was used for reverse transcription (TaqMan Micro RNA Cells-to-CT Kit, Applied Biosystems, Inc.). Briefly, biopsied embryos were placed individually into 7 μl of Cells-to-CT lysis buffer with dilute deoxyribonuclease I. After eight minutes, stop solution was added and the samples were stored at −80° C. To allow the simultaneous reverse transcription of 754 human miRNAs, three endogenous miRNA controls, and one non-human negative control for each sample, two master-mixes consisting of the A and B Megaplex RT primer pools respectively (Human Pools Set v3.0, Applied Biosystems, Inc.) were made per the manufacturer's Megaplex Pools protocol. Three μl of lysate were mixed with 4.5 μl of master-mix for each reaction for a total volume of 7.5 μl. Thermal-cycling conditions were as follows: 40 cycles at 16° C. for 2 min, 42° C. for 1 min, and 50° C. for 1 s, then 85° C. for 5 min and held at 4° C. Samples were stored at −80° C.

Pre-Amplification.

Two and a half μl of the reverse transcription product per A and B primer pool set were pre-amplified using TaqMan PreAmp Master Mix (2×) and Megaplex PreAmp Primers (10×) (Applied Biosystems, Inc.). The total reaction volume was 25 μl under these thermal-cycling conditions: an initial step of 95° C. for 10 min, 55° C. for 2 min, and 72° C. for two min followed by 12 cycles of 95° C. for 15 sec and 60° C. for four minutes. The reaction was terminated at 99° C. for 10 minutes and held at 4° C. The final pre-amplified product was not diluted.

Array PCR.

Pre-amplified RT products from individual embryos and blank media controls were profiled using two 384-well TaqMan Low Density Array (TLDA) microfluidic cards with a final dilution of 1:100 (Human miRNA A+B Cards Set v3.0, Applied Biosystems, Inc.). The arrays were loaded per the manufacturer's instructions with a total of 9 μl of preamplification product per TLDA card. The PCR was performed with TaqMan Universal PCR Master Mix, No AmpErase UNG on a 7900HT Fast Real-Time PCR System (Applied Biosystems, Inc.) under the following thermal-cycling conditions: 95° C. for 10 min followed by 40 cycles of 95° C. for 15 sec and 60° C. for one minute.

Array Statistical Analysis.

The TLDA real-time data were analyzed by using SDS RQ manager v2.4 and DataAssist v3.0 software (Applied Biosystems, Inc.). Array miRNA expression data were analyzed relative the global normalization package in the analysis software by calculating the median Ct value of each miRNA assay common among samples and using that value for normalization. Relative expression data was also calculated by using the least variant miRNA probes on each TLDA card (miR-302c on the A card and miR-760 on the B card). Finally, these results were compared using the least variant control probe common among both A and B cards (the non-coding small nuclear RNA (snRNA) U6). Comparisons between euploid and aneuploid embryos and male and female embryos were carried out using the ΔCt method where fold change was expressed as 2^{-ΔΔCt} (10). Statistical significance of fold changes was made by performing a two-sample, two-tailed Student's t-test of the ΔCt values. P-values were adjusted for false discovery by using the Benjamini-Hochberg method with a statistical significance at P<0.05 (11). Outlier values between replicates were excluded using a refined Grubbs' outlier test (12). The differentially expressed miRNAs in each group were determined by the relative expression to all normalization probes used (global normalization, least variant miRNA per A & B TLDA card, and snRNA U6. MicroRNAs with significant differences in all three normalization groups were confirmed in the following validation experiments.

Confirmation of Highly Expressed and Differentially Expressed microRNA.

An expanded panel of embryos consisting of the original 14 blastocyst embryos tested by TLDA array screening plus 13 additional blastocysts prepared in the same way were examined using single qPCR assays specific to miR-372, miR-302c and miR-720. These highly expressed miRNA were chosen because they had both low average Ct values (high concentration) and showed low variability between samples in the TLDA arrays. To confirm the differential expression of miRNAs in euploid versus aneuploid embryos found by TLDA array, seven miRNAs having the greatest differential expression (miR-26b#, hsa-miR-27b, hsa-miR-141, hsa-miR-339-3p, hsa-miR-345, hsa-miR-518a-3p, and hsa-miR-1276) were analyzed by qPCR. To confirm the differential expression of miRNAs found on TLDA screening of male versus female embryos, four miRNAs and one small-nucleolar RNA (hsa-miR-31, hsa-miR-206, hsa-miR-512-5p, hsa-miR-518d-5p, and RNU48) were analyzed by qPCR. All miRNAs were expressed relative to snRNA U6 in the expanded panel of blastocysts. Quantitative real-time PCR was performed on 384-well plates using a dilution of 1:100 preamplification products in 10 μl triplicate reactions with no-template controls, TaqMan Universal PCR Master Mix, No AmpErase UNG on the 7900HT Fast Real-Time PCR System (Applied Biosystems, Inc) with the following thermal cycling conditions: 95° C. for 10 min followed by 40 cycles of 95° C. for 15 sec and 60° C. for one min. Standard efficiency curves were calculated for each miRNA probe set using serial dilutions of cDNA in a final 10 μl reaction volume. Those used for analysis had standard curve slopes within 10% of −3.32 with R2 values of 0.99 to one.

Single Assay Statistical Analysis.

The single miRNA assay real-time data were analyzed by using SDS RQ manager v2.4 and DataAssist v3.0 software (Applied Biosystems, Inc.). MiRNA expression data were analyzed relative to the expression of snRNA U6 in each of the individual blastocyst samples. U6 was chosen for normalization because of consistent expression in all samples and its presence in both the A and B Megaplex pool sets, and because differential expression results normalized with U6 were consistent with our other normalization methods.

Comparisons between experimental groups were performed using the ΔCt method where fold change was expressed as 2^{-ΔΔCt} (10). Statistical significance of fold changes was made by performing a two-sample, two-tailed Student's t-test of the ΔCt values. Differential expression was considered significant with a P<0.05.

Results

Thirteen couples donated 91 pronuclear stage embryos for this study. From these embryos 35 developed in culture to the blastocyst stage and had trophectoderm biopsies performed for aCGH determination of chromosomal makeup. Eight embryos were excluded from further analysis: three for lack of aCGH signal, four for mosaicism, and one for PCR amplification failure. A total of 14 embryos (five female, four male, and five aneuploid) were screened for miRNA expression by TLDA arrays. An additional six female, three male, and four aneuploid were used for single assay qPCR to confirm array findings. The complete chromosomal complement of the evaluated blastocysts is shown in Table 5.

TABLE 5
Chromosomal makeup of evaluated blastocyst embryos.

| Chromosome Complement | n |
|---|---|
| 46 XY | 7 |
| 46 XX | 11 |
| 46 XX (19q 12.3-qter) | 1 |
| 45 X del (X₁) | 1 |
| 47 XX; +1 del (2q 21.3-qter) | 1 |
| 45 XY −17 | 1 |
| 46 XX; del (4q 13.2-qter); del (9q 21.11 | 1 |
| 45 XX −14 | 1 |
| 47 XXY | 1 |
| 48 XXY +19 | 1 |
| 45 XY −16 | 1 |

Highly-Expressed miRNAs in Human Blastocysts.

Out of 754 miRNAs assayed by TLDA, 135 miRNAs were detected in euploid embryos using an average Ct value cutoff of 35. The top 5% most highly expressed miRNAs had Ct values of less than 28 and were consistently detected in all samples (Table 6).

TABLE 6
Top 5% Most Highly Expressed miRNA in Euploid
Embryos (HSC—human stem cells;
MED—mammalian embryo development; PP—primate placenta)

| | | Expressed | Expressed | Expressed |
|---|---|---|---|---|
| | Avg Ct | in HSC | in MED | exclusively in PP |
| miRNA | value ± S.D. | (14-16) | (6, 7, 17-19) | (C19MC)(20, 21) |
| miR-372 | 19.68 ± 1.12 | X | X | |
| miR-512-3p | 21.30 ± 1.18 | | | X |
| miR-720 | 21.41 ± 1.07 | | X | |
| miR-1274A | 23.09 ± 1.28 | | | |
| miR-320 | 24.85 ± 0.97 | X | X | |
| miR-302b | 25.09 ± 1.65 | X | | |
| miR-106a | 25.10 ± 1.51 | | X | |
| miR-17 | 25.20 ± 1.65 | X | X | |
| miR-191 | 25.22 ± 0.97 | | | |
| miR-484 | 25.36 ± 1.02 | | X | |
| miR-886-3p | 25.68 ± 1.17 | | | |
| miR-371-3p | 25.80 ± 1.31 | X | | |
| miR-302c | 26.12 ± 1.07 | X | X | |
| miR-19b | 26.15 ± 1.45 | X | | |
| miR-378 | 26.33 ± 1.00 | | | |
| miR-520c-3p | 26.49 ± 1.28 | | | X |
| miR-373 | 26.49 ± 1.59 | X | | |
| miR-146b-5p | 26.56 ± 1.30 | | X | |
| miR-520D-3p | 26.67 ± 1.53 | | | X |
| miR-302d | 26.75 ± 1.69 | X | | |
| miR-24 | 26.75 ± 1.43 | X | X | |
| miR-302a | 26.76 ± 4.18 | X | | |
| miR-367 | 26.77 ± 1.37 | X | | |
| miR-20a | 26.92 ± 1.49 | | X | |
| miR-1275 | 27.08 ± 2.34 | | | |
| miR-30c | 27.34 ± 1.24 | | X | |
| miR-200c | 27.40 ± 1.17 | X | | |
| miR-92a | 27.42 ± 1.70 | X | | |
| miR-151-3p- | 27.44 ± 1.50 | | X | |
| miR-193b | 27.60 ± 0.93 | | | |
| miR-1260 | 27.61 ± 3.89 | X | | |
| miR-1244 | 27.84 ± 1.96 | X | | |
| miR-192 | 27.95 ± 1.14 | | | |
| miR-374a | 28.00 ± 0.97 | X | | |
| miR-519a | 28.15 ± 1.49 | | | X |
| miR-454 | 28.19 ± 0.85 | | | |
| miR-517c | 28.45 ± 1.71 | | | X |
| miR-302a# | 28.45 ± 1.25 | X | | |

Expression of several of the most highly expressed miRNAs, including miR-372, miR-720, and miR-302c, was confirmed in single assay qPCR assays with the expanded cohort and the average Ct values were within 5% of the original embryo cohort's array values.

Differential Expression of miRNA in Euploid and Aneuploid Embryos.

Initial TLDA screening identified 39 miRNAs that were differentially expressed (P<0.05) between euploid (n=9) and aneuploid (n=5) embryos (Table 7).

TABLE 7
Differentially expressed miRNAs of euploid versus aneuploid embryos.

| miRNA | Fold Change (log2) |
|---|---|
| miR-106a | 1.15 |
| miR-1276 | 6.22 |
| miR-141 | 5.49 |
| miR-146b-5p | 0.61 |
| miR-148a | 4.19 |
| miR-155 | 4.01 |
| miR-17 | 1.38 |
| miR-19a | 1.40 |
| miR-19b | 0.94 |
| miR-200c | 1.08 |
| miR-20a | 1.78 |
| miR-26b# | 3.07 |
| miR-27b | 4.29 |
| miR-30b | 1.22 |
| miR-320 | 0.48 |
| miR-339-3p | 3.69 |
| miR-345 | 1.69 |
| miR-34b | 5.26 |
| miR-367 | 0.90 |
| miR-371-3p | 0.88 |
| miR-372 | 0.67 |
| miR-373 | 1.52 |
| miR-380-5p | 3.17 |
| miR-487b | −3.35 |
| miR-509-5p | 1.33 |
| miR-517c | 1.28 |
| miR-518a-3p | 3.79 |
| miR-518c | 2.81 |
| miR-518e | 1.63 |
| miR-519a | 1.15 |
| miR-522 | 2.15 |
| miR-566 | 4.08 |
| miR-590-3p | 3.45 |
| miR-597 | 4.82 |
| miR-645 | 5.00 |
| miR-660 | 2.69 |
| miR-886-3p | 0.64 |
| miR-92a | 1.73 |
| miR-93 | 3.75 |

All miRNAs listed in Table 7 met statistical significance by at least one of the three normalization methods.

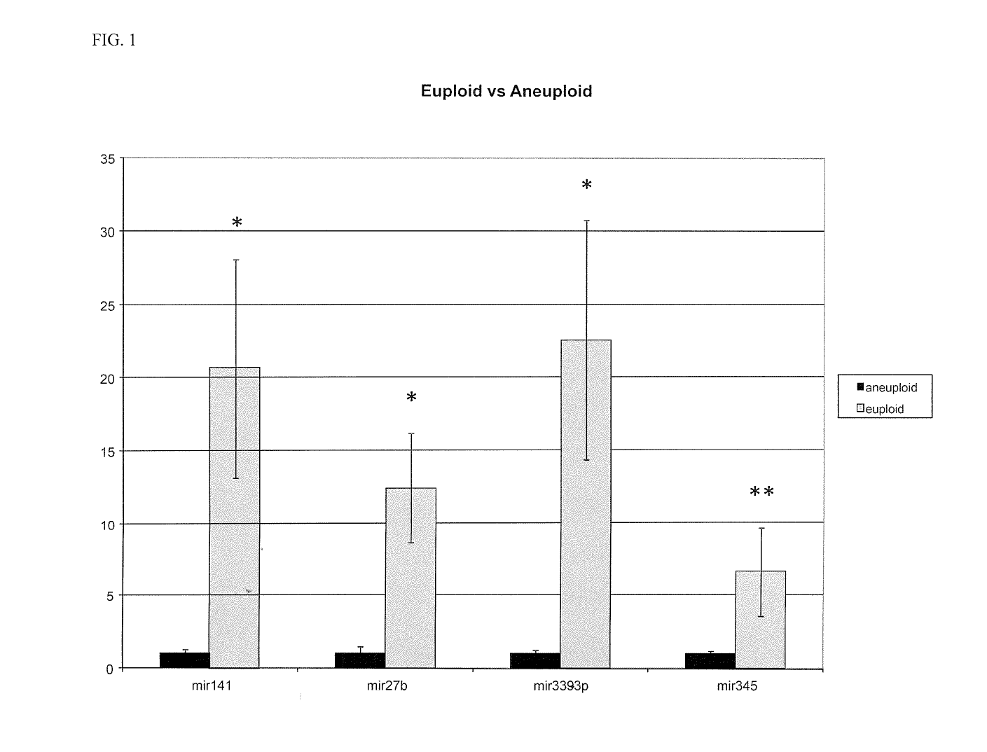

To confirm the TLDA analysis, we chose 7 miRNAs that met statistical significance using all three normalization methods and whose differential expression was at least eight-fold. All seven miRNAs chosen for validation qPCR were predicted to be more highly expressed in euploid embryos than aneuploid embryos, four of seven miRNAs were confirmed as differentially expressed (P<0.05) in the expanded cohort (FIG. 1).

Differential Expression in Male Versus Female Embryos.

Initial TLDA screening identified 21 miRNAs that were differentially expressed between male (n=4) and female (n=5) embryos (Table 8).

TABLE 8
Differentially expressed miRNAs of male versus female embryos.

| miRNA | Fold Change (log2) |
|---|---|
| miR-140-5p | 1.69 |
| miR-149 | 3.95 |
| miR-151-5p | 2.95 |
| miR-182# | −3.30 |
| miR-206 | −6.23 |
| miR-26b# | 4.02 |
| miR-31 | 2.60 |
| miR-362-3p | 3.38 |
| miR-500 | −3.84 |
| miR-512-3p | 1.21 |
| miR-512-5p | 5.89 |
| miR-518d-5p | 3.65 |
| miR-518e | 1.66 |
| miR-525-3p | 1.58 |
| miR-601 | −1.83 |
| miR-604 | −3.54 |
| miR-875-5p | −1.81 |
| miR-886-3p | 1.30 |
| miR-886-5p | 1.95 |
| miR-92a | 1.04 |
| RNU48 | 2.61 |

All miRNAs in Table 8 met statistical significance by at least one of the three normalization methods.

Four miRNAs that were separated by at least eight-fold changes were tested by qPCR in an expanded panel of 18 euploid embryos (seven male and 11 female). Of these, only miR-518d-5p was confirmed to be significantly more highly expressed in male embryos by 5.6 fold (p=0.011). Interestingly, the small-nucleolar RNA RNU48, which is often used as a control in miRNA experiments, was confirmed to be 1.7 fold more highly expressed in male embryos as well (p=0.03).

Discussion

Embryonic development is dependent on miRNAs in many species, but relatively little is known about human embryonic miRNA expression. The only prior report utilized cryopreserved blastocysts which were thawed and evaluated for a panel of 11 miRNAs known to be expressed in mouse embryos or human embryonic stem cells (13). We studied miRNA expression using more comprehensive arrays and found that human blastocysts express a large number of miRNAs. Nearly 80% of the most highly expressed miRNAs we identified (Table 6) have been previously observed to be expressed in human embryonic stem cells (14-16), developing mammalian embryos (6, 7, 17-19), or primate placentas (20, 21). We also identified miRNAs that were differentially expressed when comparing euploid to aneuploid embryos suggesting that miRNA expression is drastically altered in compromised embryos. Their role in the survival or demise of embryos, however, remains to be determined. Additionally, when comparing male to female embryos we found one miRNA that was expressed more abundantly in male embryos suggesting that some degree of sexual differentiation may be occurring even at the blastocyst stage of human development.

MicroRNA genes are organized into clusters that often allow co-expression of most of the miRNA members of the same family. The largest human cluster, extending over 100 kb, is located on chromosome 19 (C19MC) and has 46 pre-miRNA primate-specific genes that are exclusively expressed in the placenta. Interestingly, these miRNAs appear to be imprinted and are expressed only from the paternally inherited allele. Of the top 5% highly expressed miRNAs, five of them were from the C19MC cluster (Table 6). The biologic role of this cluster is unknown but could regulate some pathways of placental development and implantation. Of note, one miRNA from C19MC, mir-518d, was more highly expressed in male embryos than female embryos. MiR-518d targets doublesex/mab-3 related transcription factor 3, a gene thought to be critical in male sexual determination (22). MicroRNA sexual dimorphism has previously been observed in both murine embryonic stem cells and embryos as early as day five (23). This is the first report of a differentially expressed miRNA by gender in a human embryo.

One of the most abundant miRNA we identified in human blastocysts was miR-372. This miRNA is the human homolog of the miR-290-295 cluster in mice, which has been demonstrated to play important roles in both embryonic survival and later germ cell survival and function (6). In the mouse, miR-290-295 expression begins at the 4-8 cell stage and then decreases after embryonic day 6.5. This miRNA is abundant in the embryo and is absent in adult tissues with the exception of the gonads in both sexes. A gene knockout study of miR-290-295 resulted in reduced blastocyst development rates and a 75% decrease in fertility. The blastocysts that did survive were morphologically normal but females that were born with miR-290-295 absent were sterile due to lack of oocyte migration and subsequent ovarian failure. In addition to its importance in embryonic development, the miR-290-295 cluster is expressed in mouse embryo inner cell mass cells and these miRNAs are abundant in embryonic stem cells. Moreover, following isolation and derivation of ES cells this cluster is the first to be upregulated Expression of this family of miRNAs decreases as embryonic stem cells differentiate (6). MiR-290-295 maintains the pluripotent nature of stem cells and is involved in their rapid proliferation by promoting the GI-S transition in the cell cycle (24).

Human embryonic stem cells are known to contain unique miRNAs not found in differentiated tissues (14). Previous studies have shown that some of the stem-cell specific miRNA gene families have murine homologues that have similar genomic organizations and expression patterns. It is speculated that they may be essential in regulating the conservation of pluripotent stem cells (2). One such group, miRNA cluster 302/367, contains 5 miRNAs (mir-302a, 302b, 302c, 302d, and 367) which were all highly expressed in our study. These miRNAs have been found to catalyze the conversion of human fibroblasts into induced pluripotent stem cell (iPSC) by up to 100-fold (25, 26). It is both interesting and reassuring that many of the most highly expressed miRNAs that we found in human blastocysts were identical to those identified in human stem cells. Our data showing increased expression in the early embryo, supports a putative role for these miRNA already identified in ES cells as playing a key role in maintaining pluripotency.

We identified several miRNAs from the miR-17 family (miR106a, miR-17, miR-19b, miR20a, and miR92a) to be highly expressed in human blastocysts. The miR-17 family consists of three clusters (miR-17-92, miR-106a-363, and miR-106b-25) that have been demonstrated to be critical to mammalian development by controlling stem cell differentiation (7). Regulation of embryogenesis by these miRNAs appears to be quite complex as these miRNAs are expressed and function differently in different cell types within the embryo (27). The abundance of this group of miRNAs in our experiments suggests their importance in human embryo development.

We identified 39 and confirmed four miRNAs to be differentially expressed in human embryos according to their chromosomal makeup. We chose to confirm the most likely targets based on initial array screening. However, additional miRNAs may still differ between the two groups and should be the focus of future experiments. Out of our top potential targets, miR-141, miR-27b, miR-345, and miR-518d-5p were expressed significantly higher in euploid blastocysts when compared to aneuploid blastocysts. MiR-27b, miR-141, miR-339-3p, and miR-345 are coded on chromosomes 9, 12, 7, and 14 respectively. Two aneuploid embryos were missing a chromosome (monosomy 14) or a region of a chromosome (9q 21.11—qter) that coded for miR-345 and miR-27b respectively. Despite these deletions repeat analysis excluding these two embryos did not significantly alter fold-change outcomes and our final results remained unaffected.

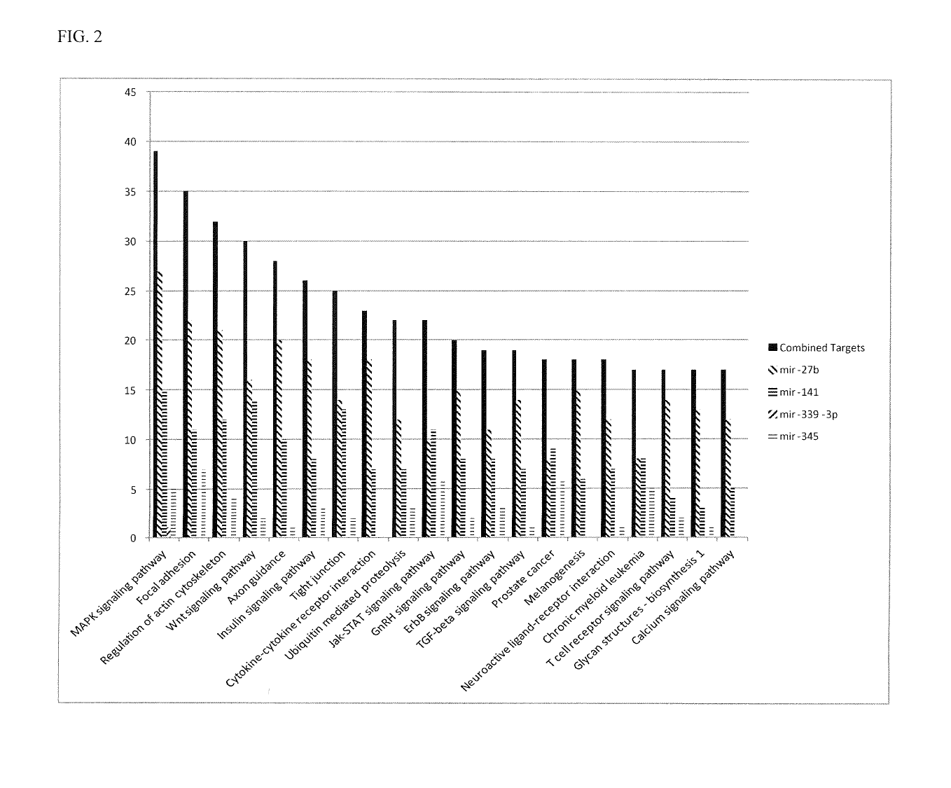

TargetScan 5 and DIANA miR-Path web-based prediction software identified 414 genes in known pathways that are targeted by these four miRNAs (28). Top predicted targets are found in FIG. 2. Many of these pathways are essential in embryo development, cell cycle, and apoptosis. MicroRNAs were initially observed to regulate apoptosis in the fruit fly by suppressing cell death (29). Newer evidence strongly supports the role of several miRNAs regulating apoptosis in mammals as well (30). In our study all of the confirmed miRNAs were expressed higher in euploid embryos. Our results are consistent with a putative role for these miRNAs in the silencing of proapoptotic genes within normal embryos or the lack of inhibition in abnormal embryos allows programmed cell death pathways to remain active.

Because of the scarcity of human embryos our sample size was limited. Despite the relatively small numbers of embryos available for our experimental groups, we were able to detect significant differences in miRNA expression between groups. Future studies utilizing larger sample sizes are necessary to confirm these results. All of the embryos used in analysis reached the blastocyst stage and met the strict criteria required to be used in clinical settings. However, due to the relatively small sample size, we were unable to stratify embryos by their exact grade or cell number. MiRNA expression likely changes rapidly throughout embryonic cell differentiation and our results could have been biased by these potential differences. Additionally, we used only a single protocol for our culturing conditions. Factors such as the source of media, protein supplementation, and pO₂ often vary significantly between embryology labs and our results may not translate well between facilities. Given these constraints, however, these experiments were performed using protocols designed for our clinical practice and, therefore, translate well to real-life situations encountered in our embryology lab. We envision miRNAs identified in this study being used as potential adjuncts to chromosomal analysis during in vitro fertilization when a cohort of varying embryos are analyzed at similar time points.

The strength of this study is that it represents the first comprehensive look at microRNA expression in human embryos. Additionally, this study is the first to show that the differential expression of miRNA based on sex or ploidy status may be occurring as early as day five of development. Human embryonic miRNA expression is robust at this stage and their putative targets are important in regulating cell proliferation, apoptosis, and differentiation. Our work provides a starting point for future studies investigating these fundamental mechanisms at the very beginning of human conception.

### REFERENCES FOR EXAMPLE II

1. Laurent L C. MicroRNAs in embryonic stem cells and early embryonic development. Journal of cellular and molecular medicine. 2008; 12(6A):2181-8. Epub 2009 Jan. 6.
2. Suh N, Blelloch R. Small RNAs in early mammalian development: from gametes to gastrulation. Development. 2011; 138(9): 1653-61. Epub 2011 Apr. 14.
3. Blakaj A, Lin H. Piecing together the mosaic of early mammalian development through microRNAs. The Journal of biological chemistry. 2008; 283(15):9505-8. Epub 2008 Feb. 15.
4. Reinhart B J, Slack F J, Basson M, Pasquinelli A E, Bettinger J C, Rougvie A E, et al. The 21-nucleotide let-7 RNA regulates developmental timing in Caenorhabditis elegans. Nature. 2000:403(6772):901-6. Epub 2000 Mar. 8.
5. Bernstein E, Kim S Y, Carmell M A, Murchison E P, Alcorn H, Li M Z, et al. Dicer is essential for mouse development. Nature genetics. 2003; 35(3):215-7. Epub 2003 Oct. 7.
6. Medeiros L A, Dennis L M, Gill M E, Houbaviy H, Markoulaki S, Fu D, et al. Mir-290-295 deficiency in mice results in partially penetrant embryonic lethality and germ cell defects. Proceedings of the National Academy of Sciences of the United States of America. 2011; 108(34):14163-8. Epub 2011 Aug. 17.
7. Foshay K M, Gallicano G I. miR-17 family miRNAs are expressed during early mammalian development and regulate stem cell differentiation. Developmental biology. 2009; 326(2):431-43. Epub 2008 Dec. 17.
8. Testart J, Lassalle B, Belaisch-Allart J, Hazout A, Forman R, Rainhorn J D, et al. High pregnancy rate after early human embryo freezing. Fertility and sterility. 1986; 46(2):268-72. Epub 1986 Aug. 1.
9. Jansen R, Mortimer D. Towards reproductive certainty: fertility & genetics beyond 1999: the plenary proceedings of the 11th World Congress on In Vitro Fertilization & Human Reproductive Genetics. Pearl River, N.Y.: Parthenon; 1999. xv, 534 p. p.
10. Schmittgen T D, Livak K J. Analyzing real-time PCR data by the comparative C(T) method. Nature protocols. 2008; 3(6): 1101-8. Epub 2008 Jun. 13.
11. Ge Y, Dudoit S, Speed T. Resampling-based multiple testing for microarray data analysis. TEST. 2003; 12(1):1-77.
12. Grubbs F E. Procedures for Detecting Outlying Observations in Samples. Technometrics. 1969; 11(1): 1-&.
13. McCallie B, Schoolcraft W B, Katz-Jaffe M G. Aberration of blastocyst microRNA expression is associated with human infertility. Fertility and sterility. 2010; 93(7):2374-82. Epub 2009 Mar. 20.
14. Suh M R, Lee Y, Kim J Y, Kim S K, Moon S H, Lee J Y, et al. Human embryonic stem cells express a unique set of microRNAs. Developmental biology. 2004; 270(2):488-98. Epub 2004 Jun. 9.
15. Morin R D, O'Connor M D, Griffith M, Kuchenbauer F, Delaney A, Prabhu A L, et al. Application of massively parallel sequencing to microRNA profiling and discovery in human embryonic stem cells. Genome research. 2008; 18(4):610-21. Epub 2008 Feb. 21.
16. Tzur G, Levy A, Meiri E, Barad O, Spector Y, Bentwich Z, et al. MicroRNA expression patterns and function in endodermal differentiation of human embryonic stem cells. PloS one. 2008; 3(11):e3726. Epub 2008 Nov. 19.
17. Fu H, Tie Y, Xu C, Zhang Z, Zhu J, Shi Y, et al. Identification of human fetal liver miRNAs by a novel method. FEBS letters. 2005; 579(17):3849-54. Epub 2005 Jun. 28.
18. Yang Y, Kai G, Pu X D, Qing K, Guo X R, Zhou X Y. Expression profile of microRNAs in fetal lung development of Sprague-Dawley rats. International journal of molecular medicine. 2012; 29(3):393-402. Epub 2011 Dec. 14.
19. Hand N J, Master Z R, Eauclaire S F, Weinblatt D E, Matthews R P, Friedman J R. The microRNA-30 family is required for vertebrate hepatobiliary development. Gastroenterology. 2009; 136(3): 1081-90. Epub 2009 Feb. 3.
20. Noguer-Dance M, Abu-Amero S, Al-Khtib M, Lefevre A, Coullin P, Moore G E, et al. The primate-specific microRNA gene cluster (C19MC) is imprinted in the placenta. Human molecular genetics. 2010; 19(18):3566-82. Epub 2010 Jul. 9.
21. Bortolin-Cavaille M L, Dance M, Weber M, Cavaille J. C19MC microRNAs are processed from introns of large Pol-II, non-protein-coding transcripts. Nucleic acids research. 2009; 37(10):3464-73. Epub 2009 Apr. 3.
22. Ottolenghi C, Fellous M, Barbieri M, McElreavey K. Novel paralogy relations among human chromosomes support a link between the phylogeny of doublesex-related genes and the evolution of sex determination. Genomics. 2002; 79(3):333-43. Epub 2002 Feb. 28.
23. Ciaudo C, Servant N, Cognat V, Sarazin A, Kieffer E, Viville S, et al. Highly dynamic and sex-specific expression of microRNAs during early ES cell differentiation. PLoS genetics. 2009; 5(8):e1000620. Epub 2009 Aug. 29.
24. Wang Y, Baskerville S, Shenoy A, Babiarz J E, Baehner L, Blelloch R. Embryonic stem cell-specific microRNAs regulate the GI-S transition and promote rapid proliferation. Nature genetics. 2008; 40(12): 1478-83. Epub 2008 Nov. 4.
25. Subramanyam D, Lamouille S, Judson R L, Liu J Y, Bucay N, Derynck R, et al. Multiple targets of miR-302 and miR-372 promote reprogramming of human fibroblasts to induced pluripotent stem cells. Nature biotechnology. 2011; 29(5):443-8. Epub 2011 Apr. 15.
26. Anokye-Danso F, Trivedi C M, Juhr D, Gupta M, Cui Z, Tian Y, et al. Highly efficient miRNA-mediated reprogramming of mouse and human somatic cells to pluripotency. Cell stem cell. 2011; 8(4):376-88. Epub 2011 Apr. 9.
27. Ventura A, Young A G, Winslow M M, Lintault L, Meissner A, Erkeland S J, et al. Targeted deletion reveals essential and overlapping functions of the miR-17 through 92 family of miRNA clusters. Cell. 2008; 132(5):875-86. Epub 2008 Mar. 11.
28. Papadopoulos G L, Alexiou P, Maragkakis M, Reczko M, Hatzigeorgiou A G. DIANA-mirPath: Integrating human and mouse microRNAs in pathways. Bioinformatics. 2009; 25(15):1991-3. Epub 2009 May 14.
29. Xu P, Vernooy S Y, Guo M, Hay B A. The Drosophila microRNA Mir-14 suppresses cell death and is required for normal fat metabolism. Current biology: CB. 2003; 13(9):790-5. Epub 2003 May 3.
30. Cimmino A, Calin G A, Fabbri M, Iorio M V, Ferracin M, Shimizu M, et al. miR-15 and miR-16 induce apoptosis by targeting BCL2. Proceedings of the National Academy of Sciences of the United States of America. 2005; 102(39):13944-9. Epub 2005 Sep. 17.

### Example III

### Human Embryos Secrete Micro RNAs into Culture Media—a Potential Biomarker for Implantation

Abstract

Objective:

To determine if human blastocysts secrete microRNAs (miRNAs) into culture media and, if so, do they reflect embryonic ploidy status and predict in vitro fertilization (IVF) outcomes after embryo transfer.

Design:

Experimental study of human embryos—IVF culture media from 15 blastocysts (five male, five female, and five aneuploid) were evaluated for miRNA expression using an array-based quantitative real time PCR (qPCR). Differentially expressed miRNAs were confirmed using qPCR in an expanded set of 28 blastocysts (eight male, eleven female, and nine aneuploid). Expression of confirmed miRNAs were also evaluated in media from 55 clinical single embryo transfer IVF cycles and correlated with pregnancy outcome.

Setting:

Academic IVF program.

Patients:

13 couples donated 91 cryopreserved embryos for this study.

Interventions:

None

Outcome Measures:

Relative miRNA expression in individual blastocysts

Results:

Ten miRNA were found to be present in IVF culture media. Only miR-191 and miR-372 were not present in media prior to embryo exposure. MiR-645 was present in the culture media prior to embryo exposure but was undetected in spent media samples. MiR-191 was more highly concentrated in media from aneuploid embryos. MiR-191, miR-372, and miR-645 were more highly concentrated in media from failed IVF cycles. Additionally, miRNAs were found to be more highly concentrated in ICSI and Day 5 media samples when compared to regularly inseminated and Day 4 samples respectively.

Conclusions:

MicroRNAs can be detected in IVF culture media. Some of these miRNAs are differentially expressed according to fertilization method, chromosomal status, and pregnancy outcome. Consequently, miRNAs are potentially good biomarkers for determining successful IVF outcomes.

Key Words:

IVF, human blastocyst, microRNA, expression, secretion, exosomes, media

Introduction

MicroRNAs (miRNAs) are small (approximately 22 nucleotides) noncoding RNAs that regulate gene expression and have been implicated in a wide array of biologic processes including early embryo development and stem cell differentiation (1). Recently, miRNAs have been found to be packaged into small vesicles called exosomes and subsequently secreted into the extracellular space (2). Encapsulated miRNAs are protected from degradation and, consequently, can be detected after extended periods of time (3). Although the role of exosomal miRNAs is still being elucidated, growing evidence suggests that packaged miRNAs can reach distant cells and affect gene expression (4).

Regardless of their physiologic role, distinct patterns of secreted miRNAs have been found to correlate with a variety of diseases including cancer (5), diabetes (6), and tissue injury (7,8). They have been detected in virtually all bodily fluids including breast milk, amniotic fluid, tears, cerebrospinal fluid, peritoneal fluid, blood, pleural fluid, saliva, semen, and urine (9). Consequently, there is great interest in identifying miRNAs within these fluids that can be used as biomarkers for the detection of diseases.

MicroRNAs are highly expressed in rapidly growing and undifferentiated cells such as cancer cells and embryonic stem cells. This led us to discover that miRNAs are highly expressed in human embryos and that intracellular miRNA expression patterns differ in euploid and aneuploid embryos (10). Since miRNAs are known to be secreted into culture media by cells grown in culture (4), the purposes of this study were to first to determine if human embryos secrete miRNAs into in vitro fertilization (IVF) culture media, and if so, to see if they were differentially secreted according to embryo chromosomal status. We further hypothesized that culture media miRNAs could be used as biomarkers to determine embryonic health prior to embryo transfer with the ultimate goal of improving live birth rates. Consequently, our final goal was to see if expression of miRNAs correlated with clinical IVF pregnancy outcomes.

Materials and Methods

Embryo Culture.

The overall study design was to screen culture media from a cohort of IVF embryos for relative and differential miRNA expression using an array-based quantitative real time PCR (qPCR) method. To confirm the miRNA array findings, the media from an expanded set of embryos was tested for miRNA expression with single miRNA qPCR assays. Finally, spent culture media from women undergoing fresh, single embryo transfer (SET) cycles were collected and analyzed for miRNA content. MicroRNA expression results from the initial experiments were then correlated to pregnancy outcomes of the SET cycles. All experiments were performed under University of Iowa Institutional Review Board (IRB) approved protocols.

Cryopreserved embryos from IVF cycles were donated for scientific research. Patients utilized IVF for a variety of infertility diagnoses (undisclosed due to IRB constraints) and had oocytes inseminated by intracytoplasmic sperm injection (ICSI). Patients utilizing standard insemination were excluded to prevent sample contamination by accessory sperm. Pronuclear stage embryos were cryopreserved by controlled rate freezing 18 to 22 hours post-ICSI in 1.5 M 1,2 propanediol (PROH; Sigma, St. Louis, Mo.) as previously described (tessart). Embryos were thawed by air warming for 40 seconds followed by 10 second exposure to 30° C. sterile water. Cryoprotectants were removed in a stepwise dilutional fashion. Surviving embryos were cultured in groups of three to four in 50 μl microdrops of IVC-One (In VitroCare; Frederick, Md.) supplemented with 20% SPS (Serum Protein Substitute, CooperSurgical Inc., Sage, Pasadena, Calif.) under oil (Cook Medical, Bloomington, Ind.) in 5.5-6.0% CO2 in air at 37° C. Embryos were moved to fresh drops of IVC-One supplemented with 20% SPS on day three. On the morning of day four, embryos were moved to individual culture in 8 μl of IVC-Three (In VitroCare) supplemented with 20% SPS. Before moving embryos to fresh drops, they were rinsed through a series of five wash drops. All embryos were cultured to the blastocyst stage and were graded according a standardized classification system (11). Embryos that had reached at least the early blastocyst stage and had an inner cell mass grade of at least a B were chosen for assisted hatching. Only hatching blastocysts were then selected for subsequent biopsy (n=15). On the morning a day five, 6 μl of spent culture media from these embryos were collected and stored at −80° C. for miRNA analysis.

To determine the chromosomal makeup of donated embryos a 10-μm channel was opened in the zona pellucida with a series of three to five laser pulses of 5 milliseconds duration (Octax Microscience, GmbH). On day five to six of culture, approximately five herniating trophectoderm (TE) cells per embryo were aspirated into a biopsy pipette and detached by firing several pulses at the area of constriction. After several passes through a wash solution, the biopsied cells were placed into a polymerase chain reaction tube with lysis buffer supplied by the Genesis Genetics Institute. The biopsies then were shipped on dry ice to Genesis Genetics Institute for array comparative genomic hybridization (aCGH) by their standard proprietary diagnostic technique.

miRNA Isolation and Detection.

In order to maximize the total amount of RNA available from each spent media sample collected, the direct Cells-to-Ct method was used for reverse transcription (TaqMan Micro RNA Cells-to-CT Kit, Applied Biosystems, Inc.). The 6 μl of day five spent media collected from each sample were placed into an equal amount of Cells-to-CT lysis buffer with dilute deoxyribonuclease I. After eight minutes, stop solution was added and the samples were stored at −80° C. To allow the simultaneous reverse transcription of 754 human miRNAs, three endogenous miRNA controls, and one non-human negative control for each sample, two master-mixes consisting of the A and B Megaplex RT primer pools respectively (Human Pools Set v3.0, Applied Biosystems, Inc.) were made per the manufacturer's Megaplex Pools protocol. Three ill of lysate were mixed with 4.5 μl of master-mix for each reaction for a total volume of 7.5 μl. Thermal-cycling conditions were as follows: 40 cycles at 16° C. for 2 min, 42° C. for 1 min, and 50° C. for 1 s, then 85° C. for 5 min and held at 4° C. Samples were stored at −80° C.

Two and a half μl of the reverse transcription product per A and B primer pool set were pre-amplified using TaqMan PreAmp Master Mix (2×) and Megaplex PreAmp Primers (10×) (Applied Biosystems, Inc.). The total reaction volume was 25 μl under these thermal-cycling conditions: an initial step of 95° C. for 10 min, 55° C. for 2 min, and 72° C. for two min followed by 12 cycles of 95° C. for 15 sec and 60° C. for four minutes. The reaction was terminated at 99° C. for 10 minutes and held at 4° C. The final pre-amplified product was not diluted.

Pre-amplified RT products from the day five spent media were profiled using two 384-well TaqMan Low Density Array (TLDA) microfluidic cards with a final dilution of 1:16 (Human miRNA A+B Cards Set v3.0, Applied Biosystems, Inc.). Two blank media samples (not exposed to embryo culture) were also analyzed by TLDA. The arrays were loaded with a total of 50 μl of preamplification product per TLDA card. The PCR was performed with TaqMan Universal PCR Master Mix, No AmpErase UNG on a 7900HT Fast Real-Time PCR System (Applied Biosystems, Inc.) under the following thermal-cycling conditions: 95° C. for 10 min followed by 40 cycles of 95° C. for 15 sec and 60° C. for one minute.

Array Statistical Analysis.

The TLDA real-time data were analyzed by using SDS RQ manager v2.4 and DataAssist v3.0 software (Applied Biosystems, Inc.). Array miRNA expression data were analyzed relative to the expression of the small nuclear RNA (snRNA) U6 control probe that was previously validated (10). Comparisons between experimental groups were carried out using the ΔCt method where fold change was expressed as 2^{-ΔΔCt}. Statistical significance of fold changes was made by performing a two-sample, two-tailed Student's t-test of the ΔCt values. The differentially expressed miRNAs in each group were determined by the relative expression to snRNA U6. MicroRNAs with significant differences were confirmed in the following experiments.

Confirmation of Differentially Expressed microRNA.

To confirm the array findings in the previous experiments, an additional group (n=13) of donated cryopreserved embryos were thaw, cultured, and biopsied for chromosome analysis as previously described. Six μl of day five media from these blastocysts were also collected for miRNA analysis and placed in an equal amount of Cells-to-Ct lysis buffer. The lysates of these additional samples were added to the original group of media samples to form an extended panel consisting of 28 day five media sample lysates. These were then processed for miRNA isolation, reverse transcribed and preamplified as previously described. To confirm the differential expression of miRNAs in euploid versus aneuploid embryo media samples found by TLDA array, miRNAs having the greatest differential expression were analyzed by qPCR. All miRNAs were expressed relative to snRNA U6 in the expanded panel of blastocyst media. As a control, blank culture media (not exposed to human embryos) both with and without added SPS protein supplement was analyzed in an identical manner as the test media. Quantitative real-time PCR was performed on 384-well plates using a dilution of 1:30 preamplification products in 10 μl triplicate reactions with no-template controls, TaqMan Universal PCR Master Mix, No AmpErase UNG on the 7900HT Fast Real-Time PCR System (Applied Biosystems, Inc) with the following thermal cycling conditions: 95° C. for 10 min followed by 40 cycles of 95° C. for 15 sec and 60° C. for one min. Standard efficiency curves were calculated for each miRNA probe set using serial dilutions of cDNA in a final 10 μl reaction volume,

Single Assay Statistical Analysis.

The single miRNA assay real-time data were analyzed by using SDS RQ manager v2.4 and DataAssist v3.0 software (Applied Biosystems, Inc.). MiRNA expression data were analyzed relative to the expression of snRNA U6 in each of the individual blastocyst media samples. U6 was chosen for normalization because of consistent expression in all media samples and its presence in both the A and 13 Megaplex pool sets.

Comparisons between experimental groups were performed using the ΔCt method where fold change was expressed as 2^{-ΔΔCt}. Statistical significance of fold changes was made by performing a two-sample, two-tailed Student's t-test of the ΔCt values. Differential expression was considered significant with a P<0.05.

Fresh SET IVF Cycles.

Expression of identified miRNAs from the previous experiments were examined in clinical IVF media samples and then correlated with IVF pregnancy outcomes. A total of 55 patients undergoing fresh single embryo transfer (SET) IVF cycles were prospectively consented to collect and analyze spent media droplets from their blastocyst culture. Patients underwent standard controlled ovarian stimulation followed by ultrasound guided oocyte retrieval. Oocytes were fertilized either by ICSI or regular insemination as determined by standard clinic protocol. Embryos were cultured in microdrops under light mineral oil in an environment of 5% to 6% CO₂ in air at 37° C. as follows: day 1 embryos were cultured in groups of 4 in 50-μL drops of IVC-One medium (InVitroCare) supplemented with 20% serum protein substitute (Cooper Surgical Inc.) for 48 hours. Day 3 embryos were transferred to individual 15-μL drops of IVC-One and 10% serum protein substitute for 24 hours. Day 4 embryos were moved to individual 15-μL drops of IVC-Three with 10% serum protein substitute for 24 hours. Before moving embryos to fresh drops, they were rinsed through five wash drops. Twelve μL of embryo-conditioned (spent) media from individual culture drops were collected on days four and five and stored at −80° C. For miRNA analysis 6 μL of spent media were thawed and processed using the same Cells-to-Ct isolation, reverse transcription, and preamplification as previously described. MicroRNA expression was determined my qPCR and single miRNA assays as described in the confirmation step above.

Results

Thirteen couples donated 91 embryos cryopreserved at the pronuclear stage for this study. From these embryos 35 developed in culture to the blastocyst stage and had trophectoderm biopsies performed for aCGH determination of chromosomal makeup. Seven embryos were excluded from further analysis: three for lack of aCGH signal and four for mosaicism. A total of 15 embryos (five female, five male, and five aneuploid) had spent media collected and screened for miRNA expression by TLDA arrays. An additional six female, three male, and four aneuploid embryos had spent media collected for single assay qPCR to confirm array findings. The complete chromosomal complement of the 28 evaluated blastocysts is shown in Table 9.

TABLE 9
Chromosomal makeup of evaluated blastocyst embryos

| Chromosome Complement | n |
|---|---|
| 46 XY | 8 |
| 46 XX | 11 |
| 46 XX (19q 12.3-qter) | 1 |
| 45 X del (X₁) | 1 |
| 47 XX; +1 del (2q 21.3-qter) | 1 |
| 45 XY −17 | 1 |
| 46 XX; del (4q 13.2-qter) | 1 |
| del (9q 21.11-qter) | |
| 45 XX −14 | 1 |
| 47 XXY | 1 |
| 48 XXY +19 | 1 |
| 45 XY −16 | 1 |

Micro-RNA Expression in IVF Culture Media of Donated Research Embryos

By TLDA screening, we consistently detected 10 miRNAs in the spent IVF culture media using a CT value cutoff of 38 as shown in Table 10.

TABLE 10
miRNA found in media

| | Average Ct Value | Average Ct |
|---|---|---|
| Assay | (spent media) | (blank media) |
| hsa-miR-106b-4373155 | 28.6 | 28.4 |
| hsa-miR-191-4395410 | 36.3 | 40 |
| hsa-miR-30c-4373060 | 24.4 | 24.6 |
| hsa-miR-372-4373029 | 25.5 | 40 |
| hsa-miR-376a-4373026 | 37.5 | 35.4 |
| hsa-miR-548a-3p-4380948 | 29.0 | 29.3 |
| hsa-miR-548c-3p-4380993 | 24.6 | 24.0 |
| hsa-miR-548d-3p-4381008 | 32.2 | 30.9 |
| hsa-miR-576-3p-4395462 | 31.3 | 30.5 |
| hsa-miR-603-001566 | 27.7 | 30.6 |
| hsa-miR-645 | 39.5 | 31.0 |

In our expanded panel of 28 embryos, only two miRNAs (miR-372 and miR-191) were confirmed to be solely in spent media samples. The rest were present in the blank media prior to embryo exposure. One miRNA, miR-645, was robustly detected in all unexposed media samples with an average CT value of 31.0 but was undetected in all of the spent media samples. Mir-191 was more highly expressed in media from aneuploid embryos than media from euploid embryos (4.7 fold, p=0.031). No differences in miRNA expression were detected when comparing media from euploid male and female embryos. To determine the source of miRNAs in media not exposed to embryos, we assayed both protein-free media and media with added serum protein substitute and only detected miRNAs in the latter.

SET Clinical Samples.

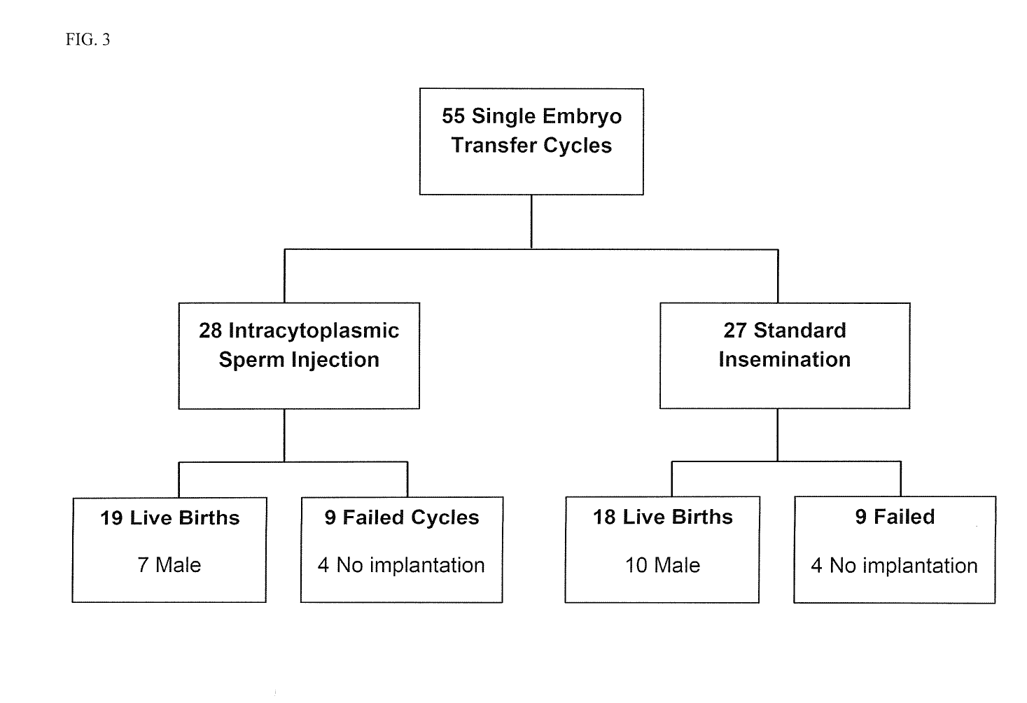

To determine if secreted miRNAs correlated with clinical IVF outcomes, media samples were collected on day 4 and day 5 of culture from patients undergoing IVF with fresh blastocyst embryo transfers. In order to easily pair media samples with pregnancy outcomes embryos were cultured in individual media droplets and patients receiving only a single embryo were included. There were a total of 55 patients recruited with cycle outcomes shown in FIG. 3. MicroRNAs previously identified to be uniquely secreted into the culture media (miR-372 and miR-191) were analyzed by single assay qPCR. MiR-645 was also investigated due the unique finding of being present in blank media samples but undetectable in conditioned media from morphologically good quality embryos.

SET Samples by Pregnancy Outcomes.

To determine if method of fertilization affected the secretion of miRNA into culture media, comparisons were made between regularly inseminated embryos and embryos fertilized by ICSI. In day 5 media miR-191 and miR-372 were found to be 4.4 fold (P=0.014) and 7.1 fold (p=0.045) more highly concentrated in media from embryos inseminated by ICSI (n=28) when compared to embryos fertilized with regular insemination (n=27). There were no differences between these two groups when day 4 media was analyzed.

SET Samples by Fertilization Method.

To determine if there were differences in miRNA secretion on different days of embryo culture, media samples from day 4 and from day 5 of embryo culture were analyzed. Both media sample groups had exposure to individual embryos for 24 hours. MiR-372 was 5.4 fold (P<0.01) more highly concentrated in day 5 media than in day 4 media. When comparing only spent media from ICSI inseminated embryos miR-191 was 1.9 fold (p=0.024) and miR-372 was 12.0 fold (P<0.01) more highly concentrated in day 5 media.

SET Samples by Day of Analysis.

Media samples from single embryo blastocyst transfers were compared between successful (live birth) samples and failed (biochemical pregnancies, spontaneous abortions, or implantation failure) samples. There were no statistically significant differences in miRNA concentrations between these groups when all the samples were analyzed. However, when analyzing media samples from embryos that were regularly inseminated only (ICSI embryos excluded), miR-191, miR-645, and miR-372 were 5.1 (p=0.018), 6.0 (p=0.024), and 7.1 (p=0.046) fold more highly detected in day 5 media from failed IVF cycle embryos (n=9) when compared to media from embryos which led to live birth (n=19).

Discussion

Selection of the best embryo for transfer during an IVF cycle is imprecise. The most commonly used selection method is to choose an embryo based on morphologic criteria. At the blastocyst stage, embryos with better morphology are more likely to have a normal chromosomal content and are more likely to implant and produce a pregnancy. However, it is known that many well-developed blastocysts of good grade will still be chromosomally abnormal or will not implant (12). Thus there is interest in finding a biomarker that will allow even better selection of the best embryo beyond morphologic criteria. Such a biomarker could lead to higher pregnancy rates per transfer and reduce the rate of multiple gestations with IVF by allowing more successful implementation of single embryo transfer.

Several potential embryonic biomarkers have been investigated recently. Several proteins secreted into the culture media have been identified and some of these have been linked to better embryo morphology (13). However, currently there are no protein biomarkers that reliably correlate with embryo implantation or pregnancy. These studies are complicated because the detection of small changes in secreted proteins is especially difficult in the background of high concentrations of protein required to be supplemented into the culture media for optimal in vitro embryo development. Metabolomic profiles have been identified in media surrounding embryos that are associated with a “healthy” embryo but, to date, this technology has not lead to an improved ability to select embryos that will implant (14,15). Pre-implantation genetic analysis of embryos has promise but it is invasive, requiring embryo biopsy. Using older methods for chromosomal analysis, this technique has not improved embryo selection as evidenced by the same or even worse pregnancy rates in prospective randomized trials (16).

The ideal biomarker would allow non-invasive analysis of the embryo by analyzing the media surrounding the embryo. The marker ideally would be stable over time, specific to the embryo, and easily and quickly measured to allow rapid assessment prior to embryo transfer. We hypothesized that microRNA (miRNA) might be such a biomarker since they are present in extracellular fluid following secretion in exosomes, are stable over time and have been shown to be potential biomarkers in plasma for a variety of conditions (5-8). Our objective was to characterize the miRNA content of media around human blastocysts and search for differential expression of miRNAs based on the genetic makeup of the embryo. We further sought to determine if miRNA concentration correlated with blastocyst implantation.

The fact that miRNA are very stable, resistant to degradation, consistently expressed, easily detected, and correlate to a variety biological processes make them, in many ways, an ideal biomarker (17).

We found miRNAs to be readily detectable in IVF culture media. However, the majority of miRNAs detected were also present in the culture media prior to embryo culture. Further analysis showed that the miRNAs were derived from the protein supplement used in our culture media. Since miRNA containing exosomes are 30-90 nm in diameter, it is feasible that they readily pass through the 200 nm filtration process the manufacturer utilizes for sterilization. This is particularly intriguing considering recent findings that miRNAs packaged into exosomes can target and affect gene transcription in remote cells (4, 18). One of the miRNAs detected in this study, miR-645, was present and robustly expressed in all blank media samples. However, miR-645 was found to be undetectable in the media from several healthy embryos. Furthermore, higher levels of miR-645 correlated to poor pregnancy outcomes in our clinical group. Taken together, this provides evidence that miRNAs may be taken up and utilized by developing embryos. Future studies could confirm this finding and determine if IVF culture media enriched or deprived of specific miRNAs could improve embryonic development.

The two other miRNAs which correlated to IVF pregnancy outcome, miR-191 and miR-372, were not present in IVF culture media prior to exposure to the embryos. Higher levels of miR-191 correlated with both aneuploid media samples and failed IVF cycles suggesting that miR-191 may be a good biomarker of embryo aneuploidy and subsequent pregnancy failure. High levels of miR-372 also correlated with IVF failure. However, miR-372 did not correlate to embryonic ploidy status. MiR-372 is known to be highly expressed in embryonic stem cells and has been recently found to be the most highly expressed miRNA in human embryos (10). The exact role these miRNAs play within embryo development has yet to be elucidated. However, target prediction software reveals miR-191 and miR372 both may regulate mitogen-activated protein kinase 1 (MAP3K1) and cyclin-dependent kinase 6 (CDK6), genes critical in cell cycle, signaling, and apoptotic pathways (Diana lab MicroT v4.0).

MicroRNAs 372 and 191 were found to be higher in the media of embryos fertilized by ICSI when compared to embryos regularly inseminated. Possible explanations could be that physical damage to the zona pellucida after ICSI permits the leakage of miRNAs into the extracellular space. MicroRNAs have been found to be more highly expressed under conditions of cell stress (19). MiR-21 is induced in endothelial cells by shear stress and modulates apoptosis and eNOS activity) Embryos fertilized by ICSI have already endured physical insult that could possibly mediate higher levels of miRNA expression. Regardless of the mechanism, when designing future studies, method of fertilization will be need to be taken into consideration, Although we found miRNAs that correlated with pregnancy outcomes, we could only find this correlation from embryos that were regularly inseminated suggesting that the process of ICSI alters miRNA secretion patterns and confounds the ability to use them as biomarkers when this method of fertilization is used.

We found miRNAs that are secreted from IVF embryos into culture media correlate to embryonic aneuploidy and pregnancy outcomes. We were hopeful to replicate our findings in day four media samples in order to be able to test embryos before a day five blastocyst transfer. However, miRNA expression was significantly lower on day four, making robust detection of secretion more difficult, perhaps leading to an inability to detect differences on this day of culture. Nevertheless, miRNA secretion is an attractive candidate for a biomarker of successful pregnancy after IVF and needs to be further investigated in larger clinical trials.

### REFERENCES FOR EXAMPLE III

1. Laurent L C. MicroRNAs in embryonic stem cells and early embryonic development. J Cell Mol Med 2008; 12(6A):2181-8. Review. PubMed PMID: 19120702.
2. Valadi H, Ekström K, Bossios A, Sjöstrand M, Lee J J, Lötvall J O. Exosome-mediated transfer of mRNAs and microRNAs is a novel mechanism of genetic exchange between cells. Nat Cell Biol 2007; 9:654-9.
3. Jung M, Schaefer A, Steiner I, Kempkensteffen C, Stephan C, Erbersdobler A, Jung K. Robust MicroRNA stability in degraded RNA preparations from human tissue and cell samples. Clin Chem 2010; 56(6):998-1006.
4. Hergenreider E, Heydt S, Treguer K, Boettger T, Horrevoets A J G, Zeiher A M, et al. Atheroprotective communication between endothelial cells and smooth muscle cells through miRNAs. Nat Cell Biol 2012; 3:249-56.
5. Chen X, Ba Y, Ma L, Cai X, Yin Y, Wang K, et al. Characterization of microRNAs in serum: a novel class of biomarkers for diagnosis of cancer and other diseases. Cell Res 2008; 18(10):997-1006. PubMed PMID: 18766170.
6. Zampetaki A, Kiechl S, Drozdov I, Willeit P, Mayr U, Prokopi M, et al. Plasma microRNA profiling reveals loss of endothelial miR-126 and other microRNAs in type 2 diabetes. Circ Res. 2010; 107(6):810-7. Epub 2010 Jul. 22. PubMed PMID: 20651284.
7. Wang K, Zhang S, Marzolf B, Troisch P, Brightman A, Hu Z, et al. Circulating microRNAs, potential biomarkers for drug-induced liver injury. Proc Natl Acad Sci USA. 2009; 106(11):4402-7. Epub 2009 Feb. 25. PubMed PMID: 19246379; PubMed Central PMCID: PMC2657429.
8. Redell J B, Moore A N, Ward N H 3rd, Hergenroeder G W, Dash P K. Human traumatic brain injury alters plasma microRNA levels. J Neurotrauma. 2010; 27(12):2147-56. Epub 2010 Nov. 23. PubMed PMID: 20883153.
9. Weber J A, Baxter D H, Zhang S, Huang D Y, Huang K H, Lee M J, Galas D J, Wang K. The microRNA spectrum in 12 body fluids. Clin Chem. 2010 November; 56(11):1733-41. Epub 2010 Sep. 16. PubMed PMID: 20847327.
10. Rosenbluth E M, Shelton D N, Sparks A E T, Devor E, Christenson L, Van Voorhis B J. Human blastocyst miRNA expression. Fertil Steril 2012. In press.
11. Testart J, Lassalle B, Belaisch-Allart J, Hazout A, Forman R, Rainhorn J D, et al. High pregnancy rate after early human embryo freezing. Fertil Steril 1986; 46:268-72.
12. Alfarawati S, Goodall N N, Fragouli E, Daphnis D D, Gordon A, Griffiths T, et al. Cytogenetic analysis of human blastocysts with the use of FISH, CGH and aCGH: scientific data and technical evaluation. Hum Reprod 2011; 26(2):480-90,
13. Mains L M, Christenson L, Yang B, Sparks A E, Mathur S, Van Voorhis B J. Identification of apolipoprotein A1 in the human embryonic secretome. Fertil Steril 2011; 96(2):422-427.e2. Epub 2011 Jun. 15. PubMed PMID: 21676393.
14. Hardarson T, Ahlstro A, Rogberg L, Botros L, Hillensjo T, Westlander G, et al. Non-invasive metabolomics profiling of day 2 and 5 embryo culture medium: a prospective randomized trial. Hum Reprod 2012; 27(1):89-96.
15. Vergouw C G, Kieslinger D C, Kostelijk E H, Botros L L, Schats R, Hompes P G, et al. Day 3 embryo selection by metabolomics profiling of culture medium with near-infrared spectroscopy as an adjunct to morphology: a randomized controlled trial. Hum Reprod 2012; 27(8):2304-11.
16. Zamora S, Clavero A, Gonzalvo M C, Luna del Castillo J D D, Roldan-Nofuentes J A, Mozas J, Castillo J A. PGS-FISH in reproductive medicine and perspective directions for improvement: a systematic review. J Assist Reprod Genet 2011; 28(8):747-57. DOI: 10.1007/s10815-011-9578-9.
17. Alevizos I, Gabor G. MicroRNAs as biomarkers in rheumatic diseases. Nat Rev Rheum 2010; 6:391-8.
18. Yang M, Chen J, Su F, Yu B, Su F. Lin L, et al. Microvesicles secreted by macrophages shuttle invasion-potentiating microRNAs into breast cancer cells. Mol Can 2011; 10:117. doi:10.1186/1476-4598-10-117.
19. Weber M, Baker M B, Moore J P, Searles C D. MiR-21 is induced in endothelial cells by shear stress and modulates apoptosis and eNOS activity. Biochem Biophys Res Commun 2010; 393(4):643-8.

We claim:

1. A method for selecting an embryo that has been grown in culture media in vitro for implantation into a patient, the method comprising detecting expression of one or more miRNAs by the embryo.

2. The method of claim 1, comprising detecting intracellular expression.

3. The method of claim 1, comprising detecting extracellular expression.

4. The method of claim 1, wherein detecting comprises converting the one or more miRNAs to DNA via performing reverse transcription and amplifying the DNA via performing a polymerase chain reaction.

5. The method of claim 1, wherein the embryo has been grown in the culture media in vitro for at least about 3 days.

6. The method of claim 5, wherein the embryo has developed into a blastocyst.

7. The method of claim 1, wherein the embryo is formed from an oocyte that was fertilized via in vitro fertilization.

8. The method of claim 7, wherein the in vitro fertilization includes intracytoplasmic sperm injection (ICSI).

9. The method of claim 1, wherein the miRNA is selected from a group consisting of hsa-miR-17, hsa-miR-19, has-miR-19a, hsa-miR-19b, hsa-miR-20a, hsa-miR-24, hsa-miR-25, hsa-miR-26b, hsa-miR-26b#, hsa-miR-27b, hsa-miR-29b, hsa-miR-29b-1, hsa-miR-29b-2, hsa-miR-30a-5p, hsa-miR-30b, hsa-miR-30c, hsa-miR-30c-1, hsa-miR-30c-2, hsa-miR-31, hsa-miR-92a, hsa-miR-93, hsa-miR-106, hsa-miR-106a, hsa-miR-106b, hsa-miR-140-5p, hsa-miR-141, hsa-miR-146b-5p, has-miRNA-148a, hsa-miR-149, hsa-miR-151-3p, hsa-miR-151-5p, has-155, hsa-miR-182#, hsa-miR-191, hsa-miR-192, hsa-miR-193b, hsa-miR-200c, hsa-miR-206, hsa-miR-302a, hsa-miR-302a#, hsa-miR-302b, hsa-miR-302b#, hsa-miR-302c, hsa-miR-302d, hsa-miR-320, hsa-miR-339, hsa-miR-339-3p, hsa-miR-345, hsa-miR-346, hsa-miR-347, hsa-miR-362-3p, hsa-miR-367, hsa-miR-371-3p, hsa-miR-372, hsa-miR-373, hsa-miR-374a, hsa-miR-376a, hsa-miR-376a, hsa-miR-376a-1, hsa-miR-376a-2, hsa-miR-378, has-miR-380-5p, hsa-miR-454, hsa-miR-484, hsa-miR-487b, hsa-miR-494, hsa-miR-500, hsa-miR-509-5p, hsa-miR-512-1, hsa-miR-512-2, hsa-miR-512-3p, hsa-miR-512-5p, hsa-miR-517c, hsa-miR-518a-1, hsa-miR-518a-2, hsa-miR-518a-3p, hsa-miR-518c, hsa-miR-518d, hsa-miR-518d-5p, hsa-miR-518e, hsa-miR-519a, hsa-miR-520c-3p, hsa-miR-520D-3p, hsa-miR-520d-5p, hsa-miR-522, hsa-miR-525-3p, hsa-miR-548a-3p, hsa-miR-548c-3p, hsa-mir-548d-3p, hsa-miR-566, hsa-miR-576-3p, hsa-miR-590-3p, hsa-miR-597, hsa-miR-601, hsa-miR-603, hsa-miR-604, hsa-miR-645, hsa-miR-660, hsa-miR-720, hsa-miR-875-5p, hsa-miR-886-3p, hsa-miR-886-5p, hsa-miR-1244, hsa-miR-1260, hsa-miR-1274A, hsa-miR-1275, and hsa-miR-1276.

10. The method of claim 9, wherein the miRNA is selected from a group consisting of hsa-mir-372, hsa-mir-645, hsa-mir-191, hsa-mir-376a, and hsa-mir-645.

11. The method of claim 10, comprising detecting extracellular expression of hsa-mir-372 or hsa-mir-645.

12. The method of claim 10, comprising detecting extracellular expression of hsa-mir-372 and hsa-mir-645.

13. The method of claim 9, wherein the miRNA is selected from a group consisting of hsa-mir-141, hsa-mir-1276, hsa-mir-27b, hsa-mir-518a-3p, hsa-mir-339-3p, hsa-mir-191, hsa-mir-30c, hsa-mir-29b, and hsa-mir-192.

14. The method of claim 13, comprising detecting intracellular expression of a miRNA selected from a group consisting of hsa-mir-141, hsa-mir-1276, hsa-mir-27b, hsa-mir-518a-3p, and hsa-mir-339-3p.

15. The method of claim 13, comprising detecting extracellular expression of a miRNA selected from a group consisting of hsa-mir-191, hsa-mir-30c, hsa-mir-29b, hsa-mir-192, and hsa-mir-27b.

16. The method of claim 9, wherein the miRNA is selected from a group consisting of hsa-mir-206, hsa-mir-512-5p, hsa-mir-26b#, hsa-mir-518d-5p, and hsa-mir-31.

17. The method of claim 1, further comprising implanting the selected embryo in the uterus of the patient.

18. A method for improving viability of an embryo grown in culture media in vitro, the method comprising supplementing or depleting the culture media of one or more miRNAs and growing the embryo in the culture media.

19. A kit comprising a set of oligonucleotide reagents selected from
(a) oligonucleotide reagents for detecting each of hsa-mir-372, hsa-mir-645, hsa-mir-191, hsa-mir-376a, and hsa-mir-645;
(b) oligonucleotide reagents for detecting each of hsa-mir-141, hsa-mir-1276, hsa-mir-27b, hsa-mir-518a-3p, hsa-mir-339-3p, hsa-mir-191, hsa-mir-30c, hsa-mir-29b, and hsa-mir-192; and
(c) oligonucleotide reagents for detecting each of hsa-mir-206, hsa-mir-512-5p, hsa-mir-26b#, hsa-mir-518d-5p, and hsa-mir-31.

20. The kit of claim 19 further comprising one or more enzymes for performing any step of RT-PCR.

21. The kit of claim 19, wherein the oligonucleotide reagents comprise a label.


SEQUENCE LISTING (103 sequences)

SEQ ID NO: 1 - RNA, 23 nt, Homo sapiens
caaagugcuuacagugcagguag

SEQ ID NO: 2 - RNA, 23 nt, Homo sapiens
ugugcaaaucuaugcaaaacuga

SEQ ID NO: 3 - RNA, 23 nt, Homo sapiens
ugugcaaauccaugcaaaacuga

SEQ ID NO: 4 - RNA, 23 nt, Homo sapiens
uaaagugcuuauagugcagguag

SEQ ID NO: 5 - RNA, 21 nt, Homo sapiens
uggcucaguucagcaggaaca

SEQ ID NO: 6 - RNA, 22 nt, Homo sapiens
cauugcacuugucucggucuga

SEQ ID NO: 7 - RNA, 21 nt, Homo sapiens
uucaaguaauucaggauaggu

SEQ ID NO: 8 - RNA, 22 nt, Homo sapiens
ccuguucuccauuacuuggcuc

SEQ ID NO: 9 - RNA, 21 nt, Homo sapiens
uucacaguggcuaaguucugc

SEQ ID NO: 10 - RNA, 23 nt, Homo sapiens
uagcaccauuugaaaucaguguu

SEQ ID NO: 11 - RNA, 24 nt, Homo sapiens
gcugguuucauauggugguuuaga

SEQ ID NO: 12 - RNA, 22 nt, Homo sapiens
cugguuucacaugguggcuuag

SEQ ID NO: 13 - RNA, 22 nt, Homo sapiens
uguaaacauccucgacuggaag

SEQ ID NO: 14 - RNA, 22 nt, Homo sapiens
uguaaacauccuacacucagcu

SEQ ID NO: 15 - RNA, 23 nt, Homo sapiens
uguaaacauccuacacucucagc

SEQ ID NO: 16 - RNA, 22 nt, Homo sapiens
cugggagaggguuguuuacucc

SEQ ID NO: 17 - RNA, 22 nt, Homo sapiens
cugggagaaggcuguuuacucu

SEQ ID NO: 18 - RNA, 21 nt, Homo sapiens
aggcaagaugcuggcauagcu

SEQ ID NO: 19 - RNA, 22 nt, Homo sapiens
uauugcacuugucccggccugu

SEQ ID NO: 20 - RNA, 22 nt, Homo sapiens
aaagugcuguucgugcagguag

SEQ ID NO: 21 - RNA, 23 nt, Homo sapiens
aaaagugcuuacagugcagguag

SEQ ID NO: 22 - RNA, 21 nt, Homo sapiens
uaaagugcugacagugcagau

SEQ ID NO: 23 - RNA, 22 nt, Homo sapiens
cagugguuuuacccuaugguag

SEQ ID NO: 24 - RNA, 22 nt, Homo sapiens
uaacacugucugguaaagaugg

SEQ ID NO: 25 - RNA, 22 nt, Homo sapiens
ugagaacugaauuccauaggcu

SEQ ID NO: 26 - RNA, 22 nt, Homo sapiens
ucagugcacuacagaacuuugu

SEQ ID NO: 27 - RNA, 23 nt, Homo sapiens
ucuggcuccgugucuucacuccc

SEQ ID NO: 28 - RNA, 21 nt, Homo sapiens
cuagacugaagcuccuugagg

SEQ ID NO: 29 - RNA, 21 nt, Homo sapiens
ucgaggagcucacagucuagu

SEQ ID NO: 30 - RNA, 23 nt, Homo sapiens
uuaaugcuaaucgugauaggggu

SEQ ID NO: 31 - RNA, 21 nt, Homo sapiens
ugguucuagacuugccaacua

SEQ ID NO: 32 - RNA, 23 nt, Homo sapiens
caacggaaucccaaaagcagcug

SEQ ID NO: 33 - RNA, 21 nt, Homo sapiens
cugaccuaugaauugacagcc

SEQ ID NO: 34 - RNA, 22 nt, Homo sapiens
aacuggcccucaaagucccgcu

SEQ ID NO: 35 - RNA, 23 nt, Homo sapiens
uaauacugccggguaaugaugga

SEQ ID NO: 36 - RNA, 22 nt, Homo sapiens
uggaauguaaggaagugugugg

SEQ ID NO: 37 - RNA, 23 nt, Homo sapiens
uaagugcuuccauguuuugguga

SEQ ID NO: 38 - RNA, 23 nt, Homo sapiens
acuuaaacguggauguacuugcu

SEQ ID NO: 39 - RNA, 23 nt, Homo sapiens
uaagugcuuccauguuuuaguag

SEQ ID NO: 40 - RNA, 22 nt, Homo sapiens
acuuuaacauggaagugcuuuc

SEQ ID NO: 41 - RNA, 23 nt, Homo sapiens
uaagugcuuccauguuucagugg

SEQ ID NO: 42 - RNA, 23 nt, Homo sapiens
uaagugcuuccauguuugagugu

SEQ ID NO: 43 - RNA, 22 nt, Homo sapiens
aaaagcuggguugagagggcga

SEQ ID NO: 44 - RNA, 21 nt, Homo sapiens
ucccuguccuccaggagcuca

SEQ ID NO: 45 - RNA, 23 nt, Homo sapiens
ugagcgccucgacgacagagccg

SEQ ID NO: 46 - RNA, 22 nt, Homo sapiens
gcugacuccuaguccagggcuc

SEQ ID NO: 47 - RNA, 23 nt, Homo sapiens
ugucugcccgcaugccugccucu

SEQ ID NO: 48 - RNA, 22 nt, Homo sapiens
aacacaccuauucaaggauuca

SEQ ID NO: 49 - RNA, 22 nt, Homo sapiens
aauugcacuuuagcaaugguga

SEQ ID NO: 50 - RNA, 23 nt, Homo sapiens
aagugccgccaucuuuugagugu

SEQ ID NO: 51 - RNA, 23 nt, Homo sapiens
aaagugcugcgacauuugagcgu

SEQ ID NO: 52 - RNA, 23 nt, Homo sapiens
gaagugcuucgauuuuggggugu

SEQ ID NO: 53 - RNA, 22 nt, Homo sapiens
uuauaauacaaccugauaagug

SEQ ID NO: 54 - RNA, 21 nt, Homo sapiens
aucauagaggaaaauccacgu

SEQ ID NO: 55 - RNA, 22 nt, Homo sapiens
aucauagaggaaaauccauguu

SEQ ID NO: 56 - RNA, 22 nt, Homo sapiens
uggugggccgcagaacaugugc

SEQ ID NO: 57 - RNA, 22 nt, Homo sapiens
cuccugacuccagguccugugu

SEQ ID NO: 58 - RNA, 22 nt, Homo sapiens
ugguugaccauagaacaugcgc

SEQ ID NO: 59 - RNA, 23 nt, Homo sapiens
uagugcaauauugcuuauagggu

SEQ ID NO: 60 - RNA, 22 nt, Homo sapiens
ucaggcucaguccccucccgau

SEQ ID NO: 61 - RNA, 22 nt, Homo sapiens
aaucguacagggucauccacuu

SEQ ID NO: 62 - RNA, 22 nt, Homo sapiens
ugaaacauacacgggaaaccuc

SEQ ID NO: 63 - RNA, 23 nt, Homo sapiens
uaauccuugcuaccugggugaga

SEQ ID NO: 64 - RNA, 21 nt, Homo sapiens
uacugcagacaguggcaauca

SEQ ID NO: 65 - RNA, 22 nt, Homo sapiens
aagugcugucauagcugagguc

SEQ ID NO: 66 - RNA, 23 nt, Homo sapiens
cacucagccuugagggcacuuuc

SEQ ID NO: 67 - RNA, 22 nt, Homo sapiens
aagugcugucauagcugagguc

SEQ ID NO: 68 - RNA, 23 nt, Homo sapiens
cacucagccuugagggcacuuuc

SEQ ID NO: 69 - RNA, 22 nt, Homo sapiens
aucgugcauccuuuuagagugu

SEQ ID NO: 70 - RNA, 20 nt, Homo sapiens
cugcaaagggaagcccuuuc

SEQ ID NO: 71 - RNA, 22 nt, Homo sapiens
gaaagcgcuucccuuugcugga

SEQ ID NO: 72 - RNA, 22 nt, Homo sapiens
gaaagcgcuucccuuugcugga

SEQ ID NO: 73 - RNA, 23 nt, Homo sapiens
caaagcgcuucucuuuagagugu

SEQ ID NO: 74 - RNA, 21 nt, Homo sapiens
caaagcgcuucccuuuggagc

SEQ ID NO: 75 - RNA, 22 nt, Homo sapiens
cucuagagggaagcacuuucug

SEQ ID NO: 76 - RNA, 21 nt, Homo sapiens
aaagcgcuucccuucagagug

SEQ ID NO: 77 - RNA, 22 nt, Homo sapiens
aaagugcauccuuuuagagugu

SEQ ID NO: 78 - RNA, 22 nt, Homo sapiens
aaagugcuuccuuuuagagggu

SEQ ID NO: 79 - RNA, 22 nt, Homo sapiens
aaagugcuucucuuuggugggu

SEQ ID NO: 80 - RNA, 20 nt, Homo sapiens
cuacaaagggaagcccuuuc

SEQ ID NO: 81 - RNA, 22 nt, Homo sapiens
aaaaugguucccuuuagagugu

SEQ ID NO: 82 - RNA, 22 nt, Homo sapiens
gaaggcgcuucccuuuagagcg

SEQ ID NO: 83 - RNA, 22 nt, Homo sapiens
caaaacuggcaauuacuuuugc

SEQ ID NO: 84 - RNA, 22 nt, Homo sapiens
caaaaaucucaauuacuuuugc

SEQ ID NO: 85 - RNA, 22 nt, Homo sapiens
caaaaaccacaguuucuuuugc

SEQ ID NO: 86 - RNA, 19 nt, Homo sapiens
gggcgccugugaucccaac

SEQ ID NO: 87 - RNA, 22 nt, Homo sapiens
aagauguggaaaaauuggaauc

SEQ ID NO: 88 - RNA, 21 nt, Homo sapiens
uaauuuuauguauaagcuagu

SEQ ID NO: 89 - RNA, 22 nt, Homo sapiens
ugugucacucgaugaccacugu

SEQ ID NO: 90 - RNA, 22 nt, Homo sapiens
uggucuaggauuguuggaggag

SEQ ID NO: 91 - RNA, 22 nt, Homo sapiens
cacacacugcaauuacuuuugc

SEQ ID NO: 92 - RNA, 19 nt, Homo sapiens
aggcugcggaauucaggac

SEQ ID NO: 93 - RNA, 19 nt, Homo sapiens
ucuaggcugguacugcuga

SEQ ID NO: 94 - RNA, 22 nt, Homo sapiens
uacccauugcauaucggaguug

SEQ ID NO: 95 - RNA, 17 nt, Homo sapiens
ucucgcuggggccucca

SEQ ID NO: 96 - RNA, 22 nt, Homo sapiens
uauaccucaguuuuaucaggug

SEQ ID NO: 97 - RNA, 21 nt, Homo sapiens
cgcgggugcuuacugacccuu

SEQ ID NO: 98 - RNA, 23 nt, Homo sapiens
cgggucggaguuagcucaagcgg

SEQ ID NO: 99 - RNA, 26 nt, Homo sapiens
aaguaguugguuuguaugagaugguu

SEQ ID NO: 100 - RNA, 18 nt, Homo sapiens
aucccaccucugccacca

SEQ ID NO: 101 - RNA, 18 nt, Homo sapiens
gucccuguucaggcgcca

SEQ ID NO: 102 - RNA, 17 nt, Homo sapiens
gugggggagaggcuguc

SEQ ID NO: 103 - RNA, 20 nt, Homo sapiens
uaaagagcccuguggagaca